SEQUENCE LISTING (27 sequences)

SEQ ID NO: 1 - DNA, 4 bp, Artificial
  STR
agat

SEQ ID NO: 2 - DNA, 8 bp, Artificial
  STR
tttctttt

SEQ ID NO: 3 - DNA, 4 bp, Artificial
  STR
aatg

SEQ ID NO: 4 - DNA, 4 bp, Artificial
  STR
aatg

SEQ ID NO: 5 - DNA, 12 bp, Artificial
  STR
tctatctgtcta

SEQ ID NO: 6 - DNA, 12 bp, Artificial
  STR
tctatctgtcta

SEQ ID NO: 7 - DNA, 4 bp, Artificial
  STR
agat

SEQ ID NO: 8 - DNA, 4 bp, Artificial
  STR
gata

SEQ ID NO: 9 - DNA, 4 bp, Artificial
  STR
tctr

SEQ ID NO: 10 - DNA, 4 bp, Artificial
  STR
gata

SEQ ID NO: 11 - DNA, 4 bp, Artificial
  STR
agat

SEQ ID NO: 12 - DNA, 4 bp, Artificial
  STR
agaa

SEQ ID NO: 13 - DNA, 35 bp, Artificial
  STR
tctatctgtctatatctatcatctatccatatcta

SEQ ID NO: 14 - DNA, 24 bp, Artificial
  PRIMER
gggggtctaagagcttgtaaaaag

SEQ ID NO: 15 - DNA, 26 bp, Artificial
  PRIMER
tgtgcatctgtaagcatgtatctatc

SEQ ID NO: 16 - DNA, 24 bp, Artificial
  PRIMER
gaacacttgtcatagtttagaacg

SEQ ID NO: 17 - DNA, 22 bp, Artificial
  PRIMER
ctgaggtatcaaaaatcagagg

SEQ ID NO: 18 - DNA, 20 bp, Artificial
  PRIMER
acagaagtctgggatgtgga

SEQ ID NO: 19 - DNA, 20 bp, Artificial
  PRIMER
gcccaaaaagacagacagaa

SEQ ID NO: 20 - DNA, 20 bp, Artificial
  PRIMER
gggtgattttcctctttggt

SEQ ID NO: 21 - DNA, 20 bp, Artificial
  PRIMER
tgattccaatcatagccaca

SEQ ID NO: 22 - DNA, 21 bp, Artificial
  PRIMER
atgttggtcaggctgactatg

SEQ ID NO: 23 - DNA, 22 bp, Artificial
  PRIMER
ccacatttatcctcattgacag

SEQ ID NO: 24 - DNA, 21 bp, Artificial
  PRIMER
atgttggtcaggctgactatg

SEQ ID NO: 25 - DNA, 22 bp, Artificial
  PRIMER
tccacattttcctcattgacag

SEQ ID NO: 26 - DNA, 24 bp, Artificial
  PRIMER
gggtgattttcctctttggtatcc

SEQ ID NO: 27 - DNA, 23 bp, Artificial
  PRIMER
agtgattccaatcatagccacag

(19) United States
(12) Patent Application Publication
(10) Pub. No.: US 2003/0215845 A1
(43) Pub. Date: Nov. 20, 2003

(54) Selective extraction of DNA from groups of cells

(76) Inventor: Todd William Bille, Lorton, VA (US)

Correspondence Address: Sherry M. Knowles, King & Spalding, 45th Floor, 191 Peachtree Street, N.E., Atlanta, GA, 30303, US

(21) Appl. No.: 10/370,143

(22) Filed: Feb. 19, 2003

Related U.S. Application Data
(60) Provisional application No. 60/358,464, filed on Feb. 19, 2002.

(51) Int. Cl. C12Q 1/68

(52) U.S. Cl. 435/6; 435/270

(57) ABSTRACT

The invention is in the area of selective extraction of DNA from groups of cells. Selective lysis of a particular cell type within a cellular mixture is performed and then the mixture is separated with a filter that allows the DNA from the lysed cells to flow through the filter, while not allowing the unlysed cells to pass through, thereby selectively extracting the DNA from a particular cell type. In one specific embodiment, spermatozoa DNA can be isolated from biological samples which also contain epithelial cells. Methods and kits are also provided which allow for the sequential extraction of DNA from mixtures of cells. The DNA in the sample can be from human, animal or vegetal origin, or any combination of human, animal or vegetal DNA.

This application claims priority to U.S. Provisional Application No. 60/358,464, filed on Feb. 19, 2002.

### FIELD OF THE INVENTION

The invention is in the area of selective extraction of DNA from groups of cells. Selective lysis of a particular cell type within a cellular mixture is performed and then the mixture is separated with a filter that allows the DNA from the lysed cells to flow through the filter, while not allowing the unlysed cells to pass through, thereby selectively extracting the DNA from a particular cell type.

### BACKGROUND OF THE INVENTION

For the past fifteen years, DNA analysis has been used to aid in the identification of suspects in criminal matters. The isolation of DNA from evidence and reference samples is a crucial step in the process of DNA profiling, also known as DNA typing. The success of genetic typing procedures depends on the availability of sufficient amounts of DNA of the appropriate quality (i.e. average fragment size) and purity. The power of Polymerase Chain Reaction (PCR) procedures has made it possible to analyze biological evidence from small samples collected during the examination of a crime. Evidence left at the scene of a crime, such as blood stains, semen stains, single hairs, bone fragments, tissue from under a victim's fingernails, epithelial cells, saliva, for example, can yield small amounts of DNA, which can then be amplified by PCR. Amplification is possible as long as there is a single strand of DNA that spans the target sequence to be amplified. Specific sequences are chosen for amplification based on their polymorphic character within the population.

Creating a reliable, informative system for human identification has been long envisaged in forensic science. Currently, there are two main methods of forensic DNA typing, PCR and restriction fragment length polymorphism (RFLP), both of which are based on DNA polymorphisms. A nucleic acid polymorphism is a condition in which different nucleotide sequences can exist at a particular site in DNA. Polymorphisms at the DNA level provide information regarding the segregation pattern of parental chromosomes during the mating process and disclose a person's genetic identity and thus, become a powerful tool for DNA typing. The information extracted from a specific DNA marker can be measured by the frequencies of each allele, which are genetic variations associated with a particular segment or locus of DNA. When several markers with different alleles are being used together for a fingerprinting procedure, information is obtained from each individual marker.

The underlying principle of RFLP is that changes in the nucleotide composition of genomic DNA often result in polymorphisms of restriction fragments, thus a variation in the size of DNA fragments can be seen after cutting with restriction enzymes. In addition, insertions or deletions of nucleotides can affect the size of the restriction fragments or can result in the elimination of restriction endonuclease target sites or the creation of new restriction endonuclease target sites.

However, RFLP requires considerable amounts of DNA and long periods of time to obtain, analyze and interpret the results. Crime-scene evidence that is old or that is present in small amounts is often unsuitable for RFLP testing. Warm moist conditions can accelerate DNA degradation rendering it unsuitable for RFLP in a relatively short period of time. PCR testing often requires less DNA than RFLP testing and the DNA can be partially degraded. However, PCR still has sample size and degradation limitations. PCR tests are extremely sensitive to contaminating DNA at the crime scene and within the test laboratory. During PCR, contaminants can be amplified up to a billion times their original concentration. Contamination can influence PCR results, particularly in the absence of proper handling techniques and proper controls for contamination.

The Polymerase Chain Reaction (PCR) has been widely used since the late 1980s and has proven to be a highly efficient and sensitive method to disclose and analyze DNA polymorphisms. One type of marker commonly analyzed by PCR are STR (Short Tandem Repeats) polymorphisms. In an STR marker, the polymorphism arises from the number of repeats of short stretches of DNA. The number of repeats varies between individuals in the general population and thus provides a source for human identification at the DNA level.

Although the DNA analysis has been conducted for over 15 years, many of the initial problems encountered have not yet been overcome. For example, the selective extraction of DNA from a particular cell type within a mixture of cells obtained from crime scenes has long been considered burdensome and sometimes leads to incorrect results.

The most common type of cell mixtures are obtained from rape and murder crime scenes that involve the victim's epithelial cells and the perpetrator's sperm cells. In order to create a DNA profile from the sperm cells to aid in the identification of a suspect, sperm cell DNA must be isolated, with little or no contamination from other sources of DNA. Any contamination can introduce uncertainty in the outcome of subsequent DNA typing since PCR can detect very small amounts of DNA in a sample.

Differential extraction is a broad term used to describe several extraction methods that can be used to separate cells. Unique characteristics of sperm cells allow for the differential extraction of the epithelial cells from the sperm cells. The first differential extraction procedure was described in 1985 (Gill et al. (1985) Nature 318: 557-9). Separation of the male fraction from the victim's DNA profile removes ambiguity in the results and allows for easier interpretation of the perpetrator's DNA profile in a rape case. Although differential extraction is commonly used to separate sperm and epithelial cells, the standard protocol is a time consuming and laborious process.

The differential extraction procedure involves preferentially breaking open the female epithelial cells with an incubation in a sarkosyl/proteinase K mixture. Sperm cells are subsequently lysed by treatment with a sarkosyl/proteinase K/dithiothreitol (DTT) mixture. The DTT breaks down the protein disulfide bridges that make up sperm nuclear membranes (Gill et al. (1985) Nature 318: 557-9). Differential extraction is effective because sperm cells are strengthened with cross-linked thiol-rich proteins, which render them impervious to digestion without DTT.

Several other methods have also been reported to extract DNA from cells. Simple protein precipitation protocols have also been modified to extract DNA. For example, the addition of 6 M NaCl to a proteinase K-digested cell extract followed by vigorous shaking and centrifugation results in a simple precipitation of the proteins so that the supernatant containing the DNA portion of cell extract can then be added to a PCR reaction. A simple alkaline lysis with 0.2 M NaOH for 5 minutes at room temperature has been shown to work as well (Rudbeck and Dissing (1998) Biotechniques 25(4):588-90). QIAamp™ spin columns have also proven effective as a means of DNA extraction (Greenspoon et al. (1998) J Forensic Sci. 43(5):1024-30). Although each of these methods is somewhat effective for extracting DNA, they do not differentially extract cell types, thus a differential organic extraction method is most often used by the forensic community.

The differential organic extraction method based on preferential lysis of epithelial cells developed by Gill et al. was devised for DNA typing using the Southern Blotting method. Since it is commonly the case that biological samples contain a greater number of vaginal epithelial cells than sperm cells, Yoshida et al. ((1995) Forensic Science International 72: 25-33) modified the differential extraction protocol. Yoshida et al. were able to demonstrate that centrifugation of the mixture after the lysis of the epithelial cells allowed for the separation of the sperm cell fraction and the epithelial cell fraction prior to lysis of the sperm cells. The authors note that this two-step differential extraction method is preferable for PCR based DNA typing. This procedure is commonly used today by the FBI Laboratory and other forensic crime laboratories to isolate the female and male fractions in sexual assault cases that contain a mixture of male and female DNA.

The long series of incubations and centrifugations that are performed to separate as much of the epithelial cell DNA from the sperm cells as possible is time consuming and labor intensive since it is highly repetitive. It must be carried out many times to remove as much of the epithelial DNA from the sperm cells as possible. Thus, this current method is inefficient, and often does not produce complete separations, resulting in a final product that is contaminated with epithelial cell DNA. Subsequent typing of genetic markers often results in three or four alleles rather than the expected one or two that would result from the complete separation of cells within the mixture. Since the extracted DNA is subsequently amplified by PCR, producing millions of copies of target DNA, even small amounts of contaminating epithelial cell DNA can interfere with the results. Furthermore, this two step standard method of differential extraction requires a large amount of sample manipulation, tedious tube labeling and the potential loss of sperm cells. Moreover, when conducted on a large scale format, these issues are amplified dramatically.

Chen et al. (1998, J Forensic Sci 42: 114-8) have attempted to overcome some of these issues by utilizing a filtration method to separate sperm cells from epithelial cells. The authors disclose that sperm cells will pass through a nylon mesh filter containing pore sizes from 5-10 microns, which allows for the separation of the larger epithelial cells (which remain on the filter) from the smaller sperm cells. However, the authors note that since older epithelial cells tend to easily lyse or may have already been broken, their nuclei can pass through the filter and result in contamination of the sperm cell DNA.

PCT Publication WO 01/52968 to Millipore Corporation also discloses a physical separation method for cell mixtures by filtration. This application teaches a method for separating a mixture of cells based on size using filtration by contacting a filter that has a defined pore size and whose pores are stable under pressure with the cell mixture and forcing the cell mixture against the filter without substantially altering the pore size. The application specifically teaches the separation of sperm cells from vaginal epithelial cells using a filter having a pore size between 5 and 30 microns. This application is directed to the physical separation of smaller sperm cells from larger epithelial cells prior to DNA extraction and analysis as an alternative to the standard differential extraction technique commonly used to separate sperm and epithelial cell DNA.

Although these techniques based on the physical separation of sperm cells and epithelial cells have been available for some time, they have not been widely implemented to solve the long-felt needs raised above.

In the field of molecular biology, DNA is routinely isolated from particular cell types within a homogeneous collection of cells through a variety of chemical means. However, these inventions are directed to the isolation of DNA from a homogeneous cell population, not the selective extraction of DNA from heterogeneous cell mixtures. In fact, these types of techniques can be utilized after the sequential extraction of DNA in the current invention is conducted as a means to further purify the DNA associated with a particular cell type.

U.S. Pat. No. 6,020,186 ('186) to Henco discloses a device to isolate nucleic acids from cells wherein the filtration matrix consists of anion exchange material. This material allows the DNA to become trapped in the matrix and then eluted upon changing buffer conditions.

U.S. Pat. No. 6,277,648 ('648) to Colpan discloses a process for the isolation of molecular cell components from a fluid sample of cells, wherein the filter used to isolate the components has a pore size which decreases in the direction of sample flow.

U.S. Pat. No. 6,310,199 ('199) to Smith is directed to a pH dependent ion exchange matrix for isolating target nucleic acids.

U.S. Pat. No. 5,660,984 to Davis discloses an apparatus comprising a non-porous DNA binding anion exchange resin to aid in the separation of DNA from other cellular components.

U.S. Pat. No. 6,274,371 ('371) to Colpan discloses a process for the preparation of plasmid DNA from microorganisms.

U.S. Pat. No. 5,990,301 ('371) to Coplan discloses a process for the purification and isolation of nucleic acids, oligonucleotides, or a combination thereof, from a bacterial or virus particle source.

Other groups have attempted to separate particular cell types from heterogeneous mixtures of cells through a variety of immunological and other means.

U.S. patent application Ser. No. 20010009757 ('757) to Bischof discloses a process for the separation of biological components from heterogeneous cell populations by binding a molecule to a biological component thereby altering the sedimentation velocity of the component and separating the bound components from the unbound components by centrifugation.

U.S. Pat. No. 6,111,096 ('096) to Laugham discloses a hyperbaric, hydrostatic pressure apparatus to partition nucleic acids from heterogeneous mixtures of cell components. This invention does not allow for the separation of different types of DNA that can be associated with particular cell types within a sample.

PCT Publication No. WO/0112847 ('847) to VanDenEeckhout is directed to a method to isolate cells from a forensic sample using of species-specific, cell type-specific or individual-specific molecules such as antibodies bound to a solid support.

PCT Publication No. WO/0077251 ('251) to Greenhalgh discloses a DNA profiling method to separate sperm cells from epithelial cells in a sample by contacting the sample with antibodies specific for antigens presented on the sperm and/or epithelial cells. Once the cells have been separated the invention discloses isolation of the DNA from the cells.

It is therefore an object of the present invention to efficiently and accurately extract DNA from a particular cell type within groups of cells.

It is still another object of the present invention to provide a means to selectively extract DNA from a particular cell type within a group of cells with little contamination of DNA from other cell types within the group.

It is another object of the present invention to provide an efficient and accurate method to selectively extract DNA from sperm cells within a group of cells.

It is another object of the present invention to provide an efficient and accurate method to selectively extract DNA from sperm cells within a group of cells that contains at least sperm cells and epithelial cells.

It is a further object of the present invention to provide a kit for the efficient and accurate extraction of DNA from groups of cells.

### SUMMARY OF THE INVENTION

The current invention solves a long-felt need in the art to selectively extract DNA from one cell type in a group of cells in an efficient and accurate manner. The current invention offers several distinct advantages over standard methods, which include reduced sample manipulation, no tube labeling, greater sensitivity, and the ability to process large numbers of specimens simultaneously. This selective DNA extraction assay is applicable to any sample which contains multiple kinds of cells, and the cells can be of human (including animal) or vegetal origin or any combination thereof.

In a first step, selective lysis of a particular cell type within a cellular mixture is performed. In a second step, the DNA from the lysed cells is allowed to flow through a size exclusion filter, which has a pore size that is greater than DNA and less than the size of intact unlysed cells, thereby preventing the unlysed cells from passing through and extracting the DNA from a particular cell type.

The filtration method allows for the physical, not chemical or ionic, separation of the smaller-sized DNA from the larger-sized intact cells.

The integrity of the material that constitutes the filter should not be compromised by either the buffers or the reagents used to lyse the cells. Optionally, the filter can be contained within a well, which is open on the top and enclosed on all sides and the bottom. One example is a cylindrical well (FIG. 3). These wells can be joined together to form a plate. For example, multiple wells can be joined together to form a multi-well plate, for example a 96 well plate (FIG. 2), each well containing a filter which is suspended and allows for an open space both above and below the filter (FIG. 3). In one embodiment, the filter is removable. In another embodiment, the filtrate is removed through a pore in the container which is opened or formed when appropriate.

In one aspect of the invention, a substrate containing at least two cell types (referred to below as Cell #1 and Cell #2) is placed in a vesicle, such as a well, and a first extraction buffer (referred to as Extraction Buffer #1) is added to the well. Extraction Buffer #1 selectively lyses Cell #1, resulting in a mixture of Cell #1 DNA (FIG. 4aC), Cell #1 cellular lysate, Cell #2 and other materials, possibly including other cells. This solution is allowed to flow through a size exclusion filter (FIG. 4aA).

The size exclusion filter has pores which are larger than DNA, but smaller than intact cells. A brief centrifugation, vacuum, gravity or other means will allow the Cell #1 DNA to flow through the filter (FIG. 4aD) wherein Cell #1 DNA can then be collected and Cell #2 remains trapped on the filter (FIG. 4aE). The solution containing Cell #2 and other materials, such as other cells, can then placed into a vesicle, for example a clean well and a second extraction buffer (referred to as Extraction Buffer #2) is added, which lyses Cell #2, resulting in a mixture of Cell #2 DNA (FIG. 4aC), Cell #2 cellular lysate, possibly other cells and other materials. Optionally, this solution can be allowed to flow through to a size exclusion filter (FIG. 4aA). The filter has pores which are larger than DNA, but smaller than intact cells. A brief centrifugation, vacuum, gravity or other means causes the Cell #2 DNA to flow through the filter which allows for Cell #2 DNA to be collected.

The extraction buffers can include any appropriate reagent that can be used to achieve lysis of cells via any acceptable method or combination of methods including, but not limited to the group consisting of mechanical disruption, chemical treatment or enzymatic digestion, such as grinding, hypotonic lysis, proteinase digestion, phenol extraction, ethanol precipitation, RNAse during restriction enzyme digestion, detergent, osmotic lysis, electroporation, ultrasound, sonication, or change in ionic concentration.

In one aspect of the invention, the heterogeneous cell mixture includes human (including animal) and vegetal cells. The human (more generally animal) cells are selectively lysed via a mechanical disruption, chemical treatment, or enzymatic digestion, in a manner that does not lyse the cell wall of the vegetal cell. It is well known that vegetal cells, due to the presence of cell walls, are substantially more resistant to lysis than human (including animal) cells.

In another aspect of the invention, the heterogeneous cell mixture includes at least sperm cells and epithelial cells. This mixture can be placed on a filter within a well of a plate. A typical sperm cell-head is approximately 5-10 μm, whereas DNA is typically smaller. Thus, in one specific embodiment of the invention the pore size of the filter is less than or equal to 5 μm. The epithelial cells are selectively lysed in any manner that does not also cause the lysis of the sperm cells, for example, via the method or combination of methods including, but not limited to the group consisting of proteinase digestion, phenol extraction, ethanol precipitation, RNAse during restriction enzyme digestion, detergent, osmotic lysis, electroporation, ultrasound, sonication, or change in ionic concentration. In one example, the epithelial cells can be lysed with any solution that does not disrupt the thiol linked proteins of the sperm cell's nucleus. In one specific example, the epithelial cells can be selectively lysed by a solution containing at least Sarkosyl and proteinase K. Once the epithelial cells have been selectively lysed, the size-exclusion properties of the filter allow the epithelial cell DNA to pass through it via gravity, vacuum centrifugation, or any other means. The filter can then be removed from the well and placed in another clean well which does not contain any epithelial cell DNA. Next the sperm cells can be lysed, via a method or combination of methods including, but not limited to proteinase digestion, phenol extraction, ethanol precipitation, RNAse during restriction enzyme digestion, detergent, osmotic lysis, electroporation, ultrasound, sonication, or change in ionic concentration. In one embodiment, the sperm cells are lysed with a solution that breaks the sperm disulfide bonds while not significantly adversely affecting the sperm DNA. In one example, the solution contains at least DTT. In another embodiment, the sperm cells can be lysed with a solution that contains at least sarkosyl, proteinase K and DTT solution. Optionally, after the sperm cells have been lysed, the lysates, sperm cell DNA, and other materials may be poured over a size exclusion filter, which allows the sperm cell DNA to flow through the filter via gravity, vacuum, centrifugation or any other means. Finally, the sperm cell DNA can be collected for further purification and analysis.

Once the separate fractions containing DNA from a particular cell type have been collected, any convenient DNA profiling method can be used to further amplify, purify, concentrate or characterize the DNA. In one embodiment, DNA profiling can be achieved through the use of a PCR-based technique, such as through the use of Short Tandem Repeats as DNA markers, HLA-DQA1 loci, or Polymarker loci.

Alternatively, restriction fragment length polymorphism (RFLP) analysis can be used for DNA typing.

Thus, in one embodiment, the invention is a method of extracting DNA from a particular cell type within a heterogeneous mixture of cells comprising:

(a) providing a sample containing a heterogeneous mixture of cells that includes a first cell type;

(b) selectively lysing the first cell type within a mixture of cells;

(c) allowing the lysed mixture that includes DNA from the first cell type to flow through a size exclusion filter; and

(d) collecting the filtrate that contains the DNA.

In one embodiment of the invention, Steps (b) and (c) are carried out simultaneously so that the selective lysis of the particular cell type is performed in a well that contains a size exclusion filter. In another embodiment, steps (b) and (c) occur sequentially.

In a further embodiment of the invention, after the DNA from a particular cell type has been collected, it can be further purified, by a variety of chemical or ionic means, including, but not limited to phenol/chloroform extraction, anion exchange resins, and pH dependent matrices.

In a still further alternate embodiment of the invention, after the DNA has been purified, a DNA typing protocol can be performed via any desired DNA profiling method to further characterize the DNA. In one embodiment, DNA profiling can be achieved through the use of a PCR-based technique, such as through the use of Short Tandem Repeats as DNA markers, HLA-DQA1 loci, or polymarker loci. Alternatively, restriction fragment length polymorphism (RFLP) analysis can be used for DNA typing.

In an another embodiment of the invention, a method is contemplated for the sequential extraction of DNA from particular cell types within a heterogeneous mixture of cells comprising:

(a) providing a sample containing a heterogeneous mixture of cells that includes at least a first and second cell type;

(b) selectively lysing the first cell type within the mixture of cells;

(c) allowing the lysed mixture that includes DNA from the first cell type to flow through a size exclusion filter;

(d) collecting the filtrate that contains the DNA from the first cell type;

(e) separately collecting the intact heterogeneous mixture of cells that includes at least the second cell type;

(f) selectively lysing the second cell type within the mixture;

(g) allowing the lysed mixture that includes DNA from the second cell type to flow through a size exclusion filter; and

(h) collecting the filtrate that contains the DNA from the second cell type.

In one embodiment of the invention, Steps (b) and (c) are carried out simultaneously so that the selective lysis of the particular cell type is performed in a well that contains a size exclusion filter. In another embodiment, steps (b) and (c) occur sequentially.

In one embodiment of the invention, Steps (f) and (g) are carried out simultaneously so that the selective lysis of the particular cell type is performed in a well that contains a size exclusion filter. In another embodiment, steps (f) and (g) occur sequentially.

In a still further alternate embodiment, the extraction of DNA is sequentially performed by repeating steps (b) through (d) to extract the DNA from each cell within the mixture of any of the following human or mammalian cell types, including, but not limited to the group consisting of erythrocytes, platelets, neutrophils, lymphocytes, monocytes, eosinophils, basophils, adipocytes, chondrocytes, pancreatic islet cells, thyroid cells, parathyroid cells, parotid cells, tumor cells, neurons, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, Peritubular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, squamous epithelial cells, osteocytes, osteoblasts, osteoclasts, and epithelial cells.

In another alternate embodiment of the invention, after the DNA from a particular cell type has been collected, it can be further purified, by a variety of chemical or ionic means, including, but not limited to phenol/chloroform extraction, anion exchange resins, and pH dependent matrices.

In still another embodiment of the invention, after the DNA has been purified, a DNA typing protocol is performed via any convenient DNA profiling method to further amplify and characterize the DNA. In one embodiment, DNA profiling can be achieved through the use of a PCR-based technique, such as through the use of Short Tandem Repeats as DNA markers, HLA-DQA1 loci, or polymarker loci. Alternatively, restriction fragment length polymorphism (RFLP) analysis can be used for DNA typing.

In one specific embodiment, the invention is directed to a method of extracting DNA from a particular cell type within a heterogeneous mixture of cells comprising:

(a) providing a sample containing a heterogeneous mixture of cells that includes at least sperm cells and epithelial cells;

(b) selectively lysing the epithelial cells;

(c) allowing the lysed mixture including the epithelial cell DNA to flow through a size exclusion filter; and

(d) collecting the filtrate that contains the epithelial cell DNA.

In one embodiment of the invention, Steps (b) and (c) are carried out simultaneously so that the selective lysis of the particular cell type is performed in a well that contains a size exclusion filter. In another embodiment, steps (b) and (c) occur sequentially.

In another embodiment, the epithelial cells are lysed with any solution that does not disrupt the thiol linked proteins of the sperm cell's nucleus. In a specific embodiment, the epithelial cells are lysed with a solution containing at least Sarkosyl and proteinase K.

In a further embodiment of the invention, after the DNA from a particular cell type has been collected, it can be further purified, by a variety of chemical or ionic means, including, but not limited to phenol/chloroform extraction, anion exchange resins, and pH dependent matrices.

In a still further alternate embodiment of the invention, after the DNA has been purified, a DNA typing protocol can be performed via any desired DNA profiling method to further characterize the DNA. In one embodiment, DNA profiling can be achieved through the use of a PCR-based technique, such as through the use of Short Tandem Repeats as DNA markers, HLA-DQA1 loci, or polymarker loci. Alternatively, restriction fragment length polymorphism (RFLP) analysis can be used for DNA typing.

In an another specific embodiment of the invention, a method is contemplated for the sequential extraction of DNA from particular cell types within a heterogeneous mixture of cells comprising:

(a) providing a sample containing a heterogeneous mixture of cells that contains at least epithelial cells and sperm cells;

(b) selectively lysing the epithelial cells;

(c) allowing the lysed epithelial cells that contains the epithelial cell DNA to flow through a size exclusion filter;

(d) collecting the filtrate that contains the epithelial cell DNA;

(e) separately collecting the intact heterogeneous mixture of cells including the sperm cells from the well;

(f) selectively lysing the sperm cells;

(g) placing the sample in a well that contains a size exclusion filter;

(h) allowing the lysed sperm cells, which includes the sperm cell DNA, to flow through the filter; and

(i) collecting the filtrate that contains the sperm cell DNA.

In one embodiment of the invention, Steps (b) and (c) are carried out simultaneously so that the selective lysis of the particular cell type is performed in a well that contains a size exclusion filter. In another embodiment, steps (b) and (c) occur sequentially.

In another embodiment, the epithelial cells are lysed with any solution that does not disrupt the thiol linked proteins of the sperm cell's nucleus. In a specific embodiment, the epithelial cells are lysed with a solution containing at least Sarkosyl and proteinase K. In a further embodiment, the sperm cells are lysed with any solution hat disrupts the thiol linked proteins of the sperm cell's nucleus. In a specific embodiment, the sperm cells are lysed with a solution containing at least DTT. IN a preferred embodiment, the sperm cells are lysed with a solution containing sarkosyl, proteinase K and DTT.

In one embodiment of the invention, Steps (f) and (g) are carried out simultaneously so that the selective lysis of the particular cell type is performed in a well that contains a size exclusion filter. In another embodiment, steps (f) and (g) occur sequentially.

In a still further alternate embodiment, the extraction of DNA is sequentially performed by repeating steps (b) through (d) to extract the DNA from each cell within the mixture of any of the following human or mammalian cell types, including, but not limited to the group consisting of erythrocytes, platelets, neutrophils, lymphocytes, monocytes, eosinophils, basophils, adipocytes, chondrocytes, pancreatic islet cells, thyroid cells, parathyroid cells, parotid cells, tumor cells, neurons, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, Peritubular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, squamous epithelial cells, osteocytes, osteoblasts, osteoclasts, and epithelial cells.

In another alternate embodiment of the invention, after the DNA from a particular cell type has been collected, it can be further purified, by a variety of chemical or ionic means, including, but not limited to phenol/chloroform extraction, anion exchange resins, and pH dependent matrices.

In still another embodiment of the invention, after the DNA has been purified, a DNA typing protocol is performed via any convenient DNA profiling method to further amplify and characterize the DNA. In one embodiment, DNA profiling can be achieved through the use of a PCR-based technique, such as through the use of Short Tandem Repeats as DNA markers, HLA-DQA1 loci, or polymarker loci. Alternatively, restriction fragment length polymorphism (RFLP) analysis can be used for DNA typing.

The invention also includes a kit for the separation of male and female DNA that can include (i) wells with filters that are larger than DNA and smaller than unlysed cells, and (ii) reagents for the selective lysis of female cells followed by the lysis of male sperm cells. Alternately, the kit can include (i) wells with filters that are larger than DNA and smaller than unlysed cells, and (iii) an instruction manual to teach the user how to use the kit for the separation of male and female DNA. The kit may also include (i) wells with filters that are larger than DNA and smaller than unlysed cells, (ii) reagents for the selective lysis of female cells followed by the lysis of male sperm cells, and, optionally, (iii) an instruction manual to teach the user how to use the kit for the separation of male and female DNA.

### DESCRIPTION OF THE DRAWINGS

FIG. 1 is a schematic representation of the preparation of “swabs” to test the validity of the Sequential Extraction protocol versus the Standard method. “Pair A” refers to a known male semen donor and oral swabs from a known female.

FIG. 2 is a schematic illustration of a 96 well plate which can be used to carry out the Sequential Extraction protocol. In A the plate is viewed from the top, whereas B depicts a side view.

FIG. 3 is a schematic illustration of an individual well which contains a filter. The filter is suspended in the well to allow for an open area both above and below the filter.

FIG. 4a is a schematic illustration of the sequential extraction of DNA from a heterogeneous cell mixture containing two cell types. In Step 1 a substrate containing two cell types is placed within a well, which contains a buffer and a filter, and the two different cells dissociate from the substrate. Next, in Step 2 Extraction Buffer #1 is added to the well, which selectively lyses Cell #1, resulting in the release of Cell #1 DNA.

In Step 3, a brief centrifugation or gravity causes Cell #1 DNA to flow through the filter. Cell #1 DNA can then be collected. In FIG. 4aE Cell #2 is larger than the pore size of the filter and thus remains trapped on the filter.

FIG. 4b is a schematic illustration depicting the final steps of the Sequential Extraction protocol. In Step 4, the filter and Cell #2 are placed into a new well, then Extraction Buffer #2 is added, which lyses Cell #2, resulting in the release of Cell #2 DNA. In Step 5, a brief centrifugation or gravity causes the Cell #2 DNA to flow through the filter. Cell #2 DNA can then be collected.

### DETAILED DESCRIPTION OF THE INVENTION

The current invention solves a long-felt need in the art to selectively extract DNA from one cell type in a group of cells in an efficient and accurate manner. The current invention offers several distinct advantages over standard methods, which include reduced sample manipulation, no tube labeling, greater sensitivity, and the ability to process large numbers of specimens simultaneously. This selective DNA extraction assay is applicable to any sample which contains multiple kinds of cells containing DNA, and the DNA can be of human (including animal) or vegetal origin or any combination of human, animal or vegetal DNA.

In a first step, selective lysis of a particular cell type within a cellular mixture is performed. In a second step, the DNA from the lysed cells is allowed to flow through a size exclusion filter, which has a pore size that is greater than DNA and less than the size of unlysed cells, thereby preventing the unlysed cells from passing through and extracting the DNA from a particular cell type.

I: Definitions

The term “differential extraction” refers to extraction methods utilized to separate cells within a heterogeneous population of cells, for example, the selective lysis of epithelial cells in an epithelial-sperm cell mixture.

The term “cell mixture” refers to a heterogeneous collection of at least two or more different cell types.

The term “PCR” refers to the polymerase chain reaction used to amplify minute amounts of DNA. PCR is a technique in which cycles of denaturation, annealing with primer, and extension with DNA polymerase, are used to amplify the number of copies of a target DNA sequence by >10⁶ times.

The term “DNA fingerprinting” refers to a technique in which DNA fragments from different individuals are compared. It can be used in any species, including humans.

The term “DNA typing” refers to the determination of the genetic code variations within a sample, for example using PCR or RFLP, to create a DNA fingerprint.

The term “biological sample” refers to any specimen that contains biological material.

The term “forensic sample” refers to a sample obtained for use to address legal issues, including, but not limited to murder, rape, trauma, assault, battery, theft, burglary, other criminal matters, identity, parental or paternity testing, and mixed-up samples. It broadly refers to a material which contains biological materials such as blood, blood stains, saliva, saliva stains, skin debris, feces, feces stains, urine, sperm cells, vaginal epithelial cells, sperm epithelial cells, other epithelial cells, muscles, bone or muscle remains or mummified remains.

The term “medical sample” refers to a sample obtained to address medical issues including, but not limited to research, diagnosis, or tissue and organ transplants.

The term “short tandem repeat” (STR) refers to all sequences between 2 and 7 nucleotides long which are tandemly reiterated within the human organism. The STRs can be represented by the formula (A_{w}GₓT_{y}C_{z})ₙ where A, G, T an C represent the nucleotides which can be in any order; w, x, y and z represent the number of each nucleotide in the sequence and range between 0 and 7 with the sum of w+x+y+z ranging between 2 and 7; and n represents the number of times the sequence is tandemly repeated and is between about 5 and 50. Most of the useful polymorphisms usually occur when the sum of w+x+y+z ranges between 3 and 7 and n ranges between 5 and 40. For dimeric repeat sequences n usually ranges between 10 and 40.

II: Selective Extraction of DNA

Step 1: Solubilization of Cells

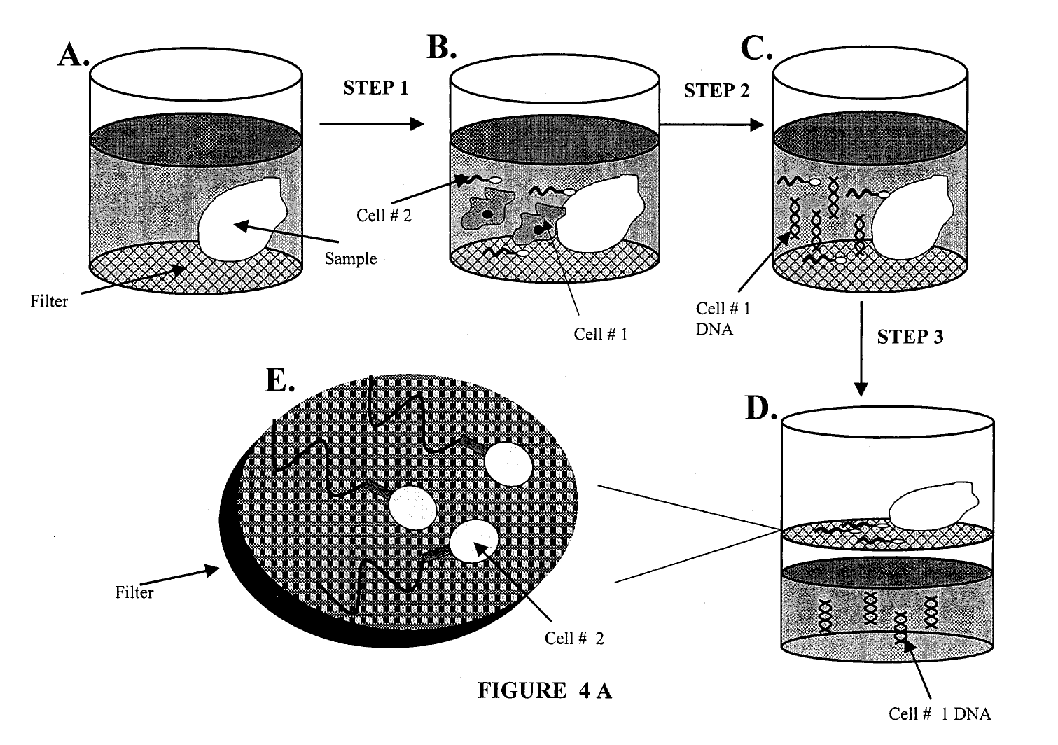

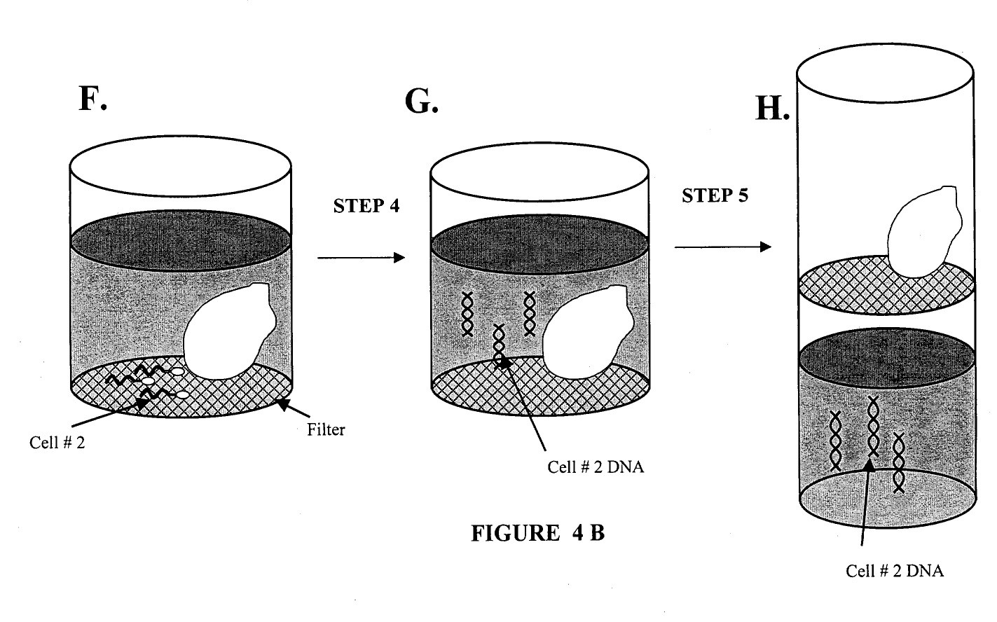

In Step 1 a sample containing at least two cell types is placed within a vesicle (FIG. 4aA), which contains buffer solution and the cells, which are dissociated from any carrier substrate (FIG. 4aB).

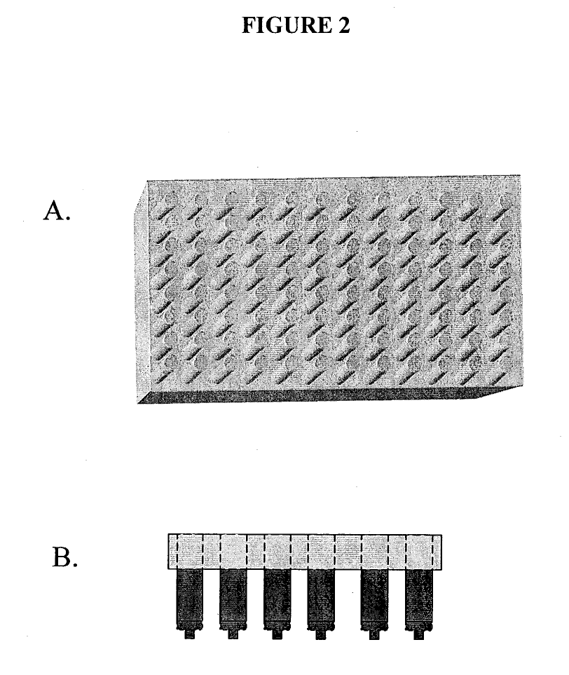

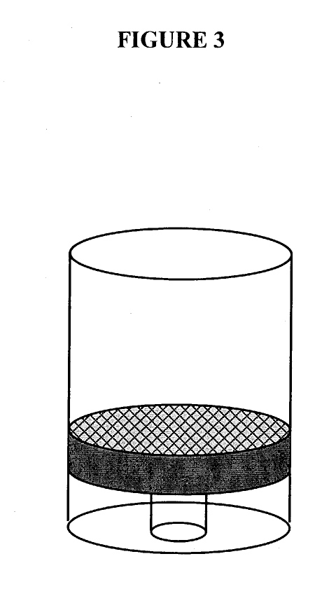

Optionally, the vesicle can be a well, which is open on the top, and enclosed on all sides and the bottom. One example is a cylindical well (FIG. 3). These wells can be joined together to form a plate. Preferably multiple wells can be joined together to form, for example, a 96 well plate (FIG. 2). Optionally, the well can contain a size exclusion filter, which is suspended and allows for an open space both above and below the filter (FIG. 3), and can be removable.

The samples can be from any source, for example, they can be biological, medical or forensic samples, including but not limited to the group consisting of cell culture, blood, semen, vaginal swabs, tissue, hair, saliva, urine, semen samples from rape victims, blood hair or semen samples from soiled clothing, identification of human remains, or any mixture of the preceding list or any mixture of body fluids.

In another embodiment, the biological, medical or forensic sample is from a human, animal or vegetal. In a specific embodiment, the sample is a vaginal swab obtained from a rape victim.

Any appropriate buffer can be used. Examples of buffers useful in the methods of the invention include, but are not limited to the following reagents or combinations of reagents: phosphate buffer solution (PBS), sodium citrate, Tris-HCl, PIPES or HEPES, Tris-HCl, Minimum Essential Medium Eagle (supplemented with or without, fetal bovine serum or basic fibroblastic growth factor (bFGF)), Neurobasal™, N2, B27, Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM—without serum), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Miniumum Essential Medium Eagle (MEM-E—with Earle's salt base), Minimum Essential Medium Eagle (MEM-H—with Hank's salt base) and Minimum Essential Medium Eagle (MEM-NAA—with non essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. These and other useful media are available from GIBCO, Grand Island, N.Y., USA and Biological Industries, Bet HaEmek, Israel, among others. A number of these media are summarized in Methods in Enzymology, Volume LVIII, “Cell Culture”, pp. 62-72, edited by William B. Jakoby and Ira H. Pastan, published by Academic Press, Inc.

Alternatively, the sample can be placed directly in an extraction (lysis) buffer that can include, for example, a reagent or combination of reagents, such as Tris-HCl, NaCl, Na₂EDTA, EGTA, SDS (sodium dodecyl sulfate), proteinase, proteinase K, TNE, N-lauroyl-sarcosine, sarkosyl, Triton, sodium pyrophosphate, glycerophosphate, leupeptin, DTT, EGTA, MgCL2, KCl, NaF, sodium valdalate, sodium molybdate, B-glycerophosphate, RIPA buffer (1% NP-40, Triton X-100, 1% sodium deoxycholate, 0.1% SDS, 0.15 molar NaCl, 0.01 molar sodium phosphate, pH 7.2, 1% Trasylol) without EDTA. NP40 buffer (1% NP-40 or Triton X-100, 0.15 molar NaCl, 0.01 molar sodium phosphate (pH 7.2), 1% Trasylol), guanidine, guanine thiocyanate or certain other chaotropic agents and detergents, ionic detergents, bile acid salts, nonionic detergents, zwitterionic detergents, alkaline lysis extraction (1 M NaCl, 1 N NaOH and/or 0.1% SDS), TWEEN 20 or a mixture of SDS or sarkosyl and Proteinase K with or without DTT.

In a further embodiment of the invention, the heterogeneous mixture of cells includes human or mammalian cells selected from, but not limited to, the group consisting of erythrocytes, platelets, neutrophils, lymphocytes, monocytes, eosinophils, basophils, adipocytes, chondrocytes, pancreatic islet cells, thyroid cells, parathyroid cells, parotid cells, tumor cells, neurons, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, Peritubular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, squamous epithelial cells, osteocytes, osteoblasts, osteoclasts, and epithelial cells.

In one embodiment of the invention, the heterogeneous mixture of cells includes at least spermatozoa and epithelial cells.

In another embodiment of the invention, the heterogeneous mixture of cells includes at least erythrocytes.

Step 2: Selective Lysis of DNA from Cell #1 in the Presence of Cell #2

In Step 2 Extraction Buffer is added to the vesicle, which can be a well. During an incubation in the extraction buffer selective lysis of Cell #1 occurs, resulting in the release of Cell #1 DNA, in the presence of Cell #2 (FIG. 4aC).

The incubation is carried out at any temperature and for any length of time that achieves the appropriate results. In one embodiment, the incubation is carried out at 37° C. for a period of time, preferably 1 or 2 hours. Alternatively, the incubation can be carried out at approximately 20-50° C. for about 30 minutes to 4 hours, or at least 1, 2, 3 or 4 hours.

In a further embodiment, the selective cell lysis can be carried out according to a method or combination of methods selected from, but not limited to, mechanical disruption, chemical treatment or enzymatic digestion, such as grinding, hypotonic lysis, proteinase digestion, phenol extraction, ethanol precipitation, RNAse during restriction enzyme digestion, detergent, osmotic lysis, electroporation, ultrasound, sonication, or change in ionic concentration. In one embodiment, the selective cell lysis can be carried with a reagent or combination of reagents selected from, but not limited to, the group consisting of Tris-HCl, NaCl, Na₂EDTA, EGTA, SDS, proteinase, proteinase K, TNE, N-lauroyl-sarcosine, sarkosyl, Triton, sodium pyrophosphate, glycerophosphate, leupeptin, DTT, EGTA, MgCL2, KCl, NaF, Sodium valdalate, sodium molybdate, B-glycerophosphate, RIPA buffer (1% NP-40, Triton X-100, 1% sodium deoxycholate, 0.1% SDS, 0.15 molar NaCl, 0.01 molar sodium phosphate, pH 7.2, 1% Trasylol) without EDTA. NP40 buffer (1% NP-40 or Triton X-100, 0.15 molar NaCl, 0.01 molar sodium phosphate (pH 7.2), 1% Trasylol), guanidine, guanine thiocyanate or certain other chaotropic agents and detergents, an alkaline lysis extraction method (1 M NaCl, 1 N NaOH and/or 0.1% SDS), TWEEN 20 or a mixture of SDS or sarkosyl and ProteinaseK with or without DTT.

In a specific embodiment of the invention, the heterogeneous mixture of cells includes at least spermatozoa and epithelial cells, and the epithelial cells are selectively lysed in the presence of sperm cells with an extraction buffer comprising at least TNE, SDS, Sarkosyl, and/or Proteinase K.

In an alternate embodiment, the heterogeneous mixture of cells includes at least spermatozoa and epithelial cells, and the sperm cells are selectively lysed in the presence of epithelial cells with an extraction buffer comprising at least DTT or any other reagent that breaks disulfide bonds. The extraction buffer can include, for example, DTT, SDS, TNE, Sarkosyl, and/or Proteinase K.

In another embodiment the heterogeneous mixture of cells contains at least erythrocytes, which can be selectively lysed in the presence of other cells. In a specific embodiment, the erythrocytes can be lysed with a solution comprising KHCO₃, NH₄Cl, and/or EDTA.

Step 3: Selective Filtration of Cell #1 DNA

In Step 3, Cell #1 DNA, Cell #1 Cellular lysates, Cell #2, and other materials, possibly other cells, are placed in a vesicle, such as a well, that contains a size exclusion filter. The filter can be suspended within the well, to allow for an open space both above and below the filter (FIG. 3), it can be a removable filter. Alternately, Steps 1-3 can be combined such that Steps 1 & 2 can be performed in a well that already contains a size-exclusion filter.

In either situation, in Step 3, Cell #1 DNA flows through the filter, while Cell #2 is larger than the pore size of the filter and thus remains trapped on the filter (FIG. 4aD-E). Cell #1 DNA can then be collected.

In one embodiment, the epithelial cell DNA flows through the filter, while the sperm cell remains trapped on the filter. Thus, in one embodiment, the filter is larger than epithelial cell DNA, but smaller than sperm cells. Sperm cell heads are typically about 25 microns, in a particular embodiment the pore size of the filter is between 5-10 microns. Alternatively, the pore size of the filter can be at least 1, 2, 3, 4, 5, 6, 7, 8, 9, 10, 11, 12, 13, 14, 15, 16, 17, 18, 19, 20, 21, 22, 23 or 24 microns, or the pore size can range from approximately 1-3, 1-4, 1-10 2-4, 2-5, 2-10, 3-5, or 3-10 microns.

In another embodiment, the filter has pores that are larger than DNA and smaller than unlysed cells. The pore size of the filter can be at least 1, 2, 3, 4, 5, 6, 7, 8, 9, 10, 11, 12, 13, 14, 15, 16, 17, 18, 19, 20, 21, 22, 23, 24, 25, 30, 40 or 50 microns. In further embodiments, the filter is removable and the filter layers are modified such that there is no affinity for nucleic acids. Still further, the filter is made of a material that is not degraded by the buffers or reagents used to perform the extraction of DNA. This material can be, for example, glass, silica, gel, titanium oxide, aluminum oxide, packed diatomaceous earth, interwoven or cemented non-wovens of glass fibers and silica gel, cellulose, paper, compressed paper, paper non-wovens, minerals bearing hydroxy groups or coated materials, such as diol-silica gel, diol-diatomaceous earth, and/or diol-perlite. The filter can be of any variety commonly used in filtering biological specimens including but not limited to microporous membranes, ultrafiltration membranes, nanofiltration membranes, or reverse osmosis membranes. Representative ultrafiltration or nanofiltration membranes include polysulphones, including polyethersulphone and polyarylsulphones, polyvinylidene fluoride, and cellulose. These membranes typically include a support layer that is generally formed of a highly porous structure. Typical materials for these support layers include various non-woven materials such as spun bounded polyethylene or polypropylene, or glass or microporous materials formed of the same or different polymer as the membrane itself. Such membranes are well known in the art, and are commercially available from a variety of sources such as Millipore Corporation of Bedford, Mass., such as the Isopore filter. In a specific embodiments, the filter can be a Qiafilter™.

In another embodiment, sample flow through the filter layer can be facilitated by applying positive or negative pressure. Due to the pore size configuration of the filter layer, passage of the sample to be filtrated through the filter layer can be driven by gravity. Furthermore, in order to accelerate the passage of sample through the filter layer, the sample can also be passed through the filter layer by centrifugation.

In one embodiment, the DNA is allowed to flow through the filter by gravity. In an alternate embodiment, the DNA is allowed to flow through the filter by centrifugation. In a specific embodiment, the centrifugation carried out for several minutes, preferably at least 3 minutes, at at least 5,600×g. Alternatively, the centrifugation can be carried out at at least 1,000, 2,000, 3,000, 4,000, 5,000, 6,000 or 7,000×g for at least 1, 2, 3, 4, 5, 6, 7, 8, 9 or 10 minutes.

Step 4: Selective Extraction of Cell #2 DNA in the Presence or Absence of Other Cells

In Step 4 Extraction Buffer is added to the well (FIG. 4bF), and during an incubation in the extraction buffer selective lyses of Cell #2 occurs, resulting in the release of Cell #2 DNA, in the presence or absence of other cells (FIG. 4bG).

In one embodiment, the incubation is carried out at approximately room temperature for a suitable period of time to achieve substantial lysis. In a specific embodiment, the incubations are carried out at 37° C. for 1-2 hours. Alternatively, the incubation can be carried out at approximately 20-50° C. for about 30 minutes to 4 hours, or at least 1, 2, 3 or 4 hours.

In another embodiment, the selective cell lysis can be carried out according to a method or combination of methods selected from, but not limited to, the group consisting of mechanical disruption, chemical treatment or enzymatic digestion, such as grinding, hypotonic lysis, proteinase digestion, phenol extraction, ethanol precipitation, RNAse during restriction enzyme digestion, detergent, osmotic lysis, electroporation, ultrasound, sonication, or change in ionic concentration. In one embodiment, the selective cell lysis can be carried with a reagent or combination of reagents selected from, but not limited to, the group consisting of Tris-HCl, NaCl, Na₂EDTA, EGTA, SDS, proteinase, proteinase K, TNE, N-lauroyl-sarcosine, sarkosyl, Triton, sodium pyrophosphate, glycerophosphate, leupeptin, SDS, DTT or other disulfide bond cleaving, EGTA, MgCL2, KCl, NaF, Sodium valdalate, sodium molybdate, B-Glycerophosphate, RIPA buffer (1% NP-40, Triton X-100, 1% sodium deoxycholate, 0.1% SDS, 0.15 molar NaCl, 0.01 molar sodium phosphate, pH 7.2, 1% Trasylol) without EDTA. NP40 buffer (1% NP-40 or Triton X-100, 0.15 molar NaCl, 0.01 molar sodium phosphate (pH 7.2), 1% Trasylol), guanidine, guanine thiocyanate or certain other chaotropic agents and detergents, an alkaline lysis extraction method (1 M NaCl, 1 N NaOH and/or 0.1% SDS), TWEEN 20 or a mixture of SDS or sarkosyl and ProteinaseK with or without DTT.

In one embodiment of the invention, the heterogeneous mixture of cells includes at least spermatozoa and epithelial cells, and the epithelial cells are selectively lysed in the presence of sperm cells with an extraction buffer comprising at least TNE, SDS, Sarkosyl, and/or Proteinase K.

In an alternate embodiment, the heterogeneous mixture of cells includes at least spermatozoa and epithelial cells, and the sperm cells are selectively lysed in the presence of epithelial cells with an extraction buffer comprising at least DTT or other disulfide cleaving agent. Alternatively, the extraction buffer can include DTT, TNE, SDS, Sarkosyl, and/or Proteinase K.

In an another embodiment, the heterogeneous mixture of cells includes at least spermatozoa and epithelial cells and the sperm cells are lysed after the epithelial cell DNA has been extracted in Steps 2 and 3 in the presence of epithelial cells with an extraction buffer comprising at least DTT.

Alternatively, the extraction buffer can include DTT, TNE, SDS, Sarkosyl, and/or Proteinase K.

In another embodiment the heterogeneous mixture of cells contains at least erythrocytes, which can be selectively lysed in the presence of other cells. In a specific embodiment, the erythrocytes can be lysed with a solution comprising KHCO₃, NH₄Cl, and/or EDTA

Step 5: Filtration of Cell #2 DNA

In one embodiment, optionally, Step 5 can be performed, in which, Cell #2 DNA flows through the size exclusion filter (FIG. 4bH). In one embodiment other cells are present in the mixture, since the pore size of the filter is smaller than unlysed cells, they will remain trapped on the filter. Cell #2 DNA can then be collected. The pore size of the filter can be at least 1, 2, 3, 4, 5, 6, 7, 8, 9, 10, 11, 12, 13, 14, 15, 16, 17, 18, 19, 20, 21, 22, 23, 24, 25, 30, 40 or 50 microns, or the pore size can range from approximately 1-3,1-4, 1-10 2-4,2-5, 2-10, 3-5, or 3-10 microns.

In one embodiment, the filter is removable and the filter layers are modified such that there is no affinity for nucleic acids. The filter should include a material that is not degraded by the buffers or reagents used to perform the extraction of DNA. This material can be, for example, glass silica gel, titanium oxide, aluminum oxide, packed diatomaceous earth, interwoven or cemented nonwovens of glass fibers and silica gel, cellulose, paper, compressed paper, paper non-wovens, minerals bearing hydroxy groups or coated materials, such as diol-silica gel, diol-diatomaceous earth, and/or diol-perlite. In another embodiment, the filter can generally be of any variety commonly used in filtering biological specimens including but not limited to microporous membranes, ultrafiltration membranes, nanofiltration membranes, or reverse osmosis membranes. Representative ultrafiltration or nanofiltration membranes include polysulphones, including polyethersulphone and polyarylsulphones, polyvinylidene fluoride, and cellulose.

These membranes typically include a support layer that is generally formed of a highly porous structure. Typical materials for these support layers include various non-woven materials such as spun bounded polyethylene or polypropylene, or glass or microporous materials formed of the same or different polymer as the membrane itself. Such membranes are well known in the art, and are commercially available from a variety of sources such as Millipore Corporation of Bedford, Mass., such as the Isopore filter. In a specific embodiments, the filter can be a Qiafilter™.

In another embodiment, sample flow through the filter layer can be facilitated by applying positive or negative pressure. Due to the pore size configuration of the filter layer, passage of the sample to be filtrated through the filter layer should be easily and conveniently be driven by gravity. Furthermore, in order to accelerate the passage of sample through the filter layer, the sample can also be passed through the filter layer by centrifugation.

In one embodiment, the DNA is allowed to flow through the filter by gravity. In an alternate embodiment, the DNA is allowed to flow through the filter by centrifugation. In a specific embodiment, the centrifugation is conducted for several minutes, preferably at least 3 minutes, at at least 5,600×g. Alternatively, the centrifugation can be carried out at at least 1,000, 2,000, 3,000, 4,000, 5,000, 6,000 or 7,000×g for at least 1, 2, 3, 4, 5, 6, 7, 8, 9 or 10 minutes.

III. DNA Isolation

After the selective extraction of DNA from a particular cell type has been achieved according to the present invention, the DNA can be isolated. DNA isolation can be achieved through a variety of chemical or ionic means.

One common method of DNA isolation is a phenol/chloroform extraction. In one embodiment, the solution used to isolate DNA contains phenol, chloroform, and/or isoamyl alcohol.

In another embodiment, a process for isolating nucleic acids is characterized by a) fixing the nucleic acids on a matrix surface; and subsequently b) eluting the nucleic acids. In one embodiment, the surface of the material forming the matrix has ion exchanging properties. Especially when using anion exchangers the nucleic acid emerging from the lysed cell can be bound reversibly to the material forming the matrix to be eluted by adjusting to high ionic strengths subsequent to various washing operations. Such a method is disclosed in U.S. Pat. No. 6,020,186.

In an alternate embodiment, the nucleic acids can be isolated according to steps comprising: (a) providing a pH dependent ion exchange matrix; (b) combining the matrix with a mixture comprising the target nucleic acid and at least one contaminant; (c) incubating the matrix and mixture at an adsorption pH, wherein the target nucleic acid adsorbs to the matrix, forming a complex; (d) separating the complex from the mixture; and (e) combining the complex with an elution solution at a desorption pH, wherein the target nucleic acid is desorbed from the complex. Such a method is disclosed in U.S. Pat. No. 6,310,199.

Other methods for the isolation of DNA will be readily apparent to one skilled in the art, including, but not limited to the boiling method (Holmes, D. S. and M. Quigley, 1981, Anal. Biochem. 114:193), the alkaline lysis method (Birnboim, H. C. and J. Doly, 1979, Nucleic Acids Res. 7:1513), cesium chloride density-gradient centrifugation, extended centrifugation steps or two phase extractions using aqueous phenol or chloroform plus ethanol precipitation and wash steps, chromatographic techniques, particularly high pressure liquid chromatography and column chromatography, DNA binding to the surface of glass and/or silicates, such as diatomaceous earth preparations or glass beads, separating DNA from mixtures containing DNA by fixing the DNA onto an anion exchange resin and removing the resin from the mixture by filtration, treating a solid material such as glass beads or silica so that its surface is coated with a hydrophilic material, such that these surfaces selectively bind proteinaceous materials and not DNA (U.S. Pat. No. 4,923,978), or using up to 100% ethyl alcohol as a binding agent to replace chaotropes typically used to facilitate binding DNA to the surface of solid particles such as silica (European Patent Application No. 0 512 767 Al).

IV. DNA Typing

Once the DNA has been isolated, various means can be used for DNA typing, such as Restriction Fragment Length Polymorphism (RFLP) Analysis and Polymerase Chain Reaction (PCR)-Based Methods, such as Short Tandem Repeat (STR) analysis and DNA amplification and typing of HLA-DQA1 loci and Polymarker loci.

Restriction fragment-length polymorphism (RFLP) analysis generates DNA fragments of different length by restriction endonucleolytic digestion. The RFLP approach entails: (i) extraction and isolation of DNA (such as that described in Steps 1-6 or some combination thereof); (ii) digestion of the DNA into fragments using a restriction endonuclease; (iii) electrophoretic separation of the fragments, based on size, for example, by agarose gel electrophoresis; (iv) denaturing the double-stranded DNA fragments, for example in a high pH environment; (v) transferring the single-stranded molecules out of the gel onto a membrane support, for example, by capillary action; (vi) hybridizing the immobilized DNA fragments with specifically labeled DNA probes; and (vii) detection of the hybrid products, for example by autoradiography or chemiluminescence.

Digestion of the DNA into Fragments Using Restriction Endonucleases

Originally, RFLP analysis was used to detect the presence or absence of specific, short DNA sequences called restriction sites. A restriction enzyme recognizes this short sequence along the double-stranded DNA and cuts the DNA wherever the specific site resides. There are three types of restriction endonucleases, Type II restriction endonucleases bind to the double stranded DNA at a particular recognition sequence and then they cleave the molecule by cutting the DNA backbones somewhere along this sequence. This type will always cut the DNA only at the specific site it recognizes. Therefore, it should produce the same DNA fragments if you use a particular DNA molecule and the same Type II enzyme for the digestion. This type has been extensively used in recombinant DNA technology. For example, the restriction enzyme HaeIII recognizes and cuts the DNA at the sequence GGCC. Other examples of restriction endonucleases include EcoRI, HindiIII, PstI, EcoRV, SfiI, SgrAI, FokI, and BspMi. Information on commercially available restriction endonucleases can be obtained from: Roberts, R. J. and Macelis, D., Nucleic Acids Res., 27, 312-313, 1999, McClelland, M., Nelson, M. and Raschke, E., Nucleic Acids Res., 22, 3640-3659, 1994., or Roberts, R. J., The Restriction Enzyme Database, New England BioLabs, Inc., REBASE version 103, 2001.

The DNA from a sample can be cut into many fragments, and due to sequence differences (i.e., in the enzyme recognition sequence among individuals), individuals can have restriction fragments of different lengths that can be used for comparisons.

There are genetic polymorphisms that exists in the human genome that do not encode proteins and are highly polymorphic. One class of these genetic markers is known as variable number tandem repeats (VNTRs) or minisatellites. The VNTRs are tandemly repeated sequences (usually 9-80 bases in length per repeat unit) that exhibit variation in the number of repeats for alleles within and among individuals. Following digestion with a restriction enzyme, the length of each fragment is determined by the number of repeats contained within each fragment. Many VNTR loci used for human identity testing exhibit more than 100 types in a population. In fact, such a high degree of polymorphism is exhibited that the typing of five to eight markers is sufficient to differentiate most, if not all, unrelated individuals. In other words, a multiple locus VNTR profile is extremely rare. More importantly, typing VNTR loci currently provides the scientist the best avenue to exclude a suspect who has been falsely associated with an evidentiary sample. In addition, typing can be accomplished, at times, with less than 50 ng of high molecular weight genomic DNA.

One factor that affects the effectiveness of RFLP analysis is the availability of well-characterized VNTR loci. The VNTR loci must be compatible with the restriction enzyme utilized for RFLP analysis (for example, HaeIII). Compatibility refers to the repeat sequence of the VNTR, which usually does not contain the restriction site specific to the restriction enzyme used in the assay. The loci alleles should generally fall in a size range that is greater than 500 bp and less than 20,000 bp. The loci routinely typed are D1S7, D2S44, D4S110, D10S28, and D17S79 (Table 1). Additionally, VNTR loci are highly polymorphic and have a high degree of sensitivity of detection.

Electrophoresis

DNA molecules, regardless of size, have the same charge-to-mass ratio. Thus, all DNA fragments separated based on charge will migrate at the same rate and cannot be resolved. Therefore, digested double-stranded DNA fragments are separated based on size by electrophoresis through a sieving medium, and the electrophoretic system is performed using submarine gels. The horizontal, agarose gels are submerged beneath buffer to maintain phase continuity and to enable effective heat dissipation in the relatively thick gels. Generally, fragments from 500 to 25,000 bp in length can be separated.

The use of polyacrylamide gel in electrophoresis (PAGE) allows for a separation or fractionation of samples on the basis of molecular size in addition to the charge differences. The separation by size is the result of the sieving effect imparted by control of the gel pore size in a “separating gel” layer.

The gels can consist of two separately polymerized layers of polyacrylamide, the separating and the stacking gel. The polymer is the result of reaction between monomer and co-monomer or cross-linking agent (percent C). The sum of the concentrations of acrylamide monomer and cross-linking agent is expressed as percent T. The separating gel has a higher concentration of monomers and consequently a smaller pore size. The actual separation of the samples takes place in this gel. The restriction created by the small pores of this gel endows PAGE with high resolution power. There can be a second gel layer with larger pore size or stacking gel to help the sample concentrate itself into tightly-packed starting zones.

The gels are placed in an electrophoretic chamber containing electrolyte buffer. The sample, generally combined with a high-density solution and a tracking dye, is placed between the gel and the buffer. The high-density solution helps the sample diffuse less. The tracking dye helps to visually follow the progress of the electrophoresis and also functions as a reference point for the measurement of the relative mobility of the bands (R_{f}). Upon application of an electrical potential, the leading ion of the separating compartment, which is chosen to have a higher effective mobility than the sample species, migrates out in front of all others, while the trailing ion of the electrolyte buffer replaces it, both moving in the same direction. Behind the leading zone other zones form, depending on the specific mobilities of the sample species, and produce discrete bands. The buffer ions and pH are important to the resolution of the macromolecular mixture to be separated and to the enzymatic activity remaining after the electrophoretic separation has occurred.

Discontinuous (disc) electrophoresis utilizing polyacrylamide as the supporting medium has been claimed as one of the most effective methods for the separation of ionic components. It employs discontinuous (multiphasic) buffers varying in chemical composition and properties on electrode wells and gels. The theory of discontinuous buffers was introduced by Omstein and Davis [Ann. N.Y. Acad. Sci., 121:320 and 404 (1964)].

Southern Blotting

Southern blotting is the transfer of the electrophoretically-separated array of digested DNA fragments out of the gel and onto a membrane support (such as nitrocellulose or nylon) (Southern et al, J. Mol. Bio. 98: 503-517 (1975)). The blotting relies on a flow, by capillary action, of a transfer solution from a reservoir through the agarose gel to a membrane overlaid by a stack of dry paper towels or blot pads. The DNA fragments are carried along with the flow of transfer solution from the gel to the membrane. Under appropriate conditions, the DNA readily binds to the membrane, maintaining the same array as it had at the end of electrophoresis. At some point before reaching the membrane, the DNA fragments must be denatured to single-stranded DNA so that the probe can bind during hybridization.

Two examples of protocols for blotting are alkali transfer and high salt transfer—an alkali transfer to a positively charged nylon membrane is compatible with autoradiographic detection; whereas a high salt transfer to a neutrally charged nylon membrane is compatible with chemiluminescent detection. A low ionic strength, alkaline environment, which enables covalent binding of DNA to charged nylon membranes, is simple to make (i.e., 0.4 M NaOH) and also denatures the DNA during transfer. In contrast, a high salt transfer system first requires a denaturation of the DNA step and then a neutralization step of the gel prior to setting up the transfer.

Membranes

The membrane should made of a material that can bind DNA efficiently, for example, nitrocellulose or nylon. Efficient DNA binding is desirable so that the target DNA will not leach off the membrane after usage. UV fixing with neutral-charged membranes or basic pH and positive-charged membranes have been used to effectively immobilize DNA to nylon. The DNA should be single stranded when bound.

Probes

Any fragment of nucleic acid can be used as a hybridization probe as long as it can be labeled so that the duplex can be detected.

The choice of probe (or probe design) depends on the typing technology, the availability of the probe, and the degree it can be labeled. DNA can be cloned into plasmids or bacteriophages. Thus, probe yield can be increased, and stability can be maintained. The vector should not contain sequences that cross-react with the target sequences of the probe. Otherwise, the vector sequences can have to be removed prior to using the probe. The use of double-stranded probes encounters two competing reactions, which are reassociation of the probe and hybridization to the immobilized DNA. Hybridization with single-stranded probes does not have to address reassociation with the probe's complement. Synthetic probes offer an alternative in that an enzyme or other molecule (e.g., biotin) can be coupled directly to the probe. The longer the probe, the greater the specificity, buy hybridization times are longer than that for shorter probes.

Probe labeling Probes are labeled either isotopically or nonisotopically. ³²P is the most commonly used radioisotope. Radioactive probes can be labeled with ³²P to a specific activity greater than 10⁵ counts per minute (cpm)/μL using commercially available labeling kits. In one example, 50- to 100-ng aliquots of probe are labeled. Prior to hybridization, the probe is denatured by boiling for several minutes followed by quenching on ice. The process of nick translation utilizes DNase I to crate single-stranded nicks in double-stranded DNA. The 5′→3′ exonuclease and 5′→3′ polymerase actions of escherichia coli DNA Polymerase I are then used to remove stretches of single-stranded DNA starting at the nicks and replace them with new strands made by the incorporation of labeled deoxyribonucleotides. As a result, each nick moves along the DNA strand and is repaired in a 5′→3′ direction. Nick translation can utilize any dNTP labeled with ³²P.

Nonradioactive labeling can allow for the incorporation of biotinylated nucleotides into DNA by standard techniques, such as nick translation or by direct labeling. Alternatively, an enzyme can be covalently linked to the probe directly or bound indirectly. Alkaline phosphatase-labeled oligonucleotide probes for VNTR loci and molecular weight markers are commercially available.

Hybridization

Hybridization is the annealing of a complementary probe to membrane-immobilized genomic target DNA (or vice versa). Basically, for RFLP typing, denatured DNA is immobilized on an inert support, such as nitrocellulose or nylon, so that it is accessible to incoming single-stranded probes. The probes are labeled to facilitate detection of the probe-target duplex.

The hybridization solution for probing VNTR sequences immobilized to nylon membranes can contain formamide, Denhardt's solution, dextran sulfate, or other additives, for example, sodium dodecyl sulfate (SDS), polyethylene glycol (PEG), and phosphate buffer.

To distinguish between similar related sequences, reaction conditions should be optimized for the application. Factors that affect hybridization rates are: length of the fragments, base composition, ionic strength (cations; stringency), viscosity, denaturing agents (used to reduce the hybridization temperature because of fragility of nitrocellulose membranes), and temperature (stringency). Single-stranded probes are favored over denatured probes because re-annealing is avoided. High probes are favored over denatured probes because re-annealing is avoided. High probe concentration drives the reaction, but too high a concentration should be avoided as it will lead to nonspecific hybridization. The rate of hybridization is decreased with increasing length of probe. The rate increases with GC content, but the effect usually is not substantial. Temperature affects hybridization rate, which is slow at low temperatures and increases to a broad range usually 20° to 25° C. below the desired melting temperature (Tₘ) for annealing. At high temperatures, the strands tend to dissociate. The use of formamide decreases the Tₘ and has been used to reduce the hybridization temperature to 35° to 45° C. At low ionic strength (low salt), DNA fragments hybridize very slowly. High salt environments tend to stabilize mismatched duplexes. Dextran sulphate can able used to increase the hybridization rate (10%-tenfold) due to exclusion of the DNA from the volume occupied by the polymer, effectively increasing the DNA concentration (probe) or by inducing probe concatenation.

Hybridization generally is carried out in plastic sandwich boxes or in roller bottles. The membranes should be completely wetted and submerged in the hybridization solution. Large air bubbles trapped next to the membrane should be avoided, as these bubbles will impede probe hybridization. Gentle shaking can occur during the process.

Post Hybridization Washes

Post-hybridization washes can be carried out to remove loosely bound probe that could lead to nonspecific membrane background staining. Wash stringency increases as the solution temperature is increased and the buffer salt concentration is decreased. As the wash stringency increases, greater amounts of mismatched probe are removed from target DNA.

Autoradiography

For DNA typing of single-copy genomic targets by RFLP, sensitivity of detection requirements often dictated that ³²P-labeled probes can be utilized. The detection of the isotopic label can be facilitated by autoradiography using high speed X-ray film. The radioactive object (generally on a membrane) normally is placed in contact with X-ray film, and the energy released from the decay products of the radioisotope is absorbed by silver halide grains in the film emulsion to form a latent image. A chemical development process amplifies the latent image and renders the image visible on the film. Because the majority of emissions from ³²P pass through the thin film emulsion with contributing to the final image, the detection process can suffer from long exposure times and lack of sensitivity. Therefore, the membrane is sandwiched between X-ray film, and this complex is sandwiched between intensifying screens and exposed at approximately −70° C.

Intensifying screens can be required to convert the high energy radiation that passes through the film to emitted light, which exposes the film in the same spatial pattern as the emissions from the radioactively labeled material.

Chemiluminescence

An alternative to the use of radioactively labeled probes is an approach that covalently links alkaline phosphatase directly to DNA probes. The annealed probe target hybrid can be detected using a variety of reagents, particularly chemiluminescence substrates.

Application of chemiluminescent detection to RFLP typing requires a system with continuous light output so that signal can be collected over time (for increased sensitivity) and, optionally, multiple exposures to film can be made. The most sensitive chemiluminescent systems are those that emit a continuous glow. These systems have been applied widely to genetic research and involve the selective cleavage of stabilized 1,2-dioxetanes. one particularly useful substrate is LUMI-PHOS Plus® (Life Technologies Gaithersburg, Md., USA). The LUMI-PHOS Plus substrate yields a continuous light output for more than 48 hours.

Detailed descriptions of protocols for RFLP analysis are further disclosed in U.S. Pat. Nos. 5,593,832 and 5,514,547, as well as in Budowle et al. (DNA Typing Protocols: Molecular Biology and Forensic Analysis, Eaton Publishing: MA, USA (2000)).

Short Tandem Repeat Loci Using Polyacrylamide Gel Electrophoresis

Short Tandem Repeat Loci

A subclass of variable number tandem repeats (VNTRs) is the short tandem repeat (STR), or microsatellite, loci. The STR loci are composed of tandemly repeated sequences, each of which is 2 to 7 bp in length. Loci containing repeat sequences consisting of 4 bp (or tetranucleotides) are used routinely for human identification and, in some cases, 5 bp repeat STRs used. These repeat sequence loci are abundant in the human genome and are highly polymorphic. The number of alleles at a tetranucleotide repeat STR locus ranges usually from 5 to 20. STR loci are amenable to amplification by PCR.

In one embodiment, loci selected from the group or combinations of the group consisting of thirteen STR loci, CSF1PO, FGA, TH01, TPOX, vWA, D3S1358, D5S818, D7S820, D8S1179, D13S317, D16S539, D18S51, and D21S11, that have been selected as the core loci for use in the national DNA databank, Combined DNA Index System CODIS (Table 1) can be used for STR typing.

TABLE 1
Thirteen CODIS STR Core Loci Characteristics

| | Chromosome | | Repeat Sequence |
|---|---|---|---|
| STR Name | Location | Gene Association | Motif |
| CSF1PO | 5q33.3-34 | CSF-1 receptor | AGAT |
| | | protooncogene | |
| FGA | 4q28 | Human alpha fibrinogen | (TTTC)₃ TTTT |
| TH01 | 11p15.5 | Tyrosine hydroxylase | (AATG)ₙ |
| TPOX | 2p23-2pter | Thyroid peroxidase | (AATG)ₙ |
| vWA | 12p12-pter | von Willebrand antigen | TCTA (TCTG)₃₋₄ |
| | | | (TCTA)ₙ |
| D3S1358 | 3p | anonymous | TCTA (TCTG)₁₋₃ |
| | | | (TCTA)ₙ |
| D5S818 | 5q21-q31 | anonymous | (AGAT)ₙ |
| D7S820 | 7q | anonymous | (GATA)ₙ |
| D8S1179 | 8 | anonymous | (TCTR)ₙ |
| D13S317 | 13q22-q31 | anonymous | (GATA)ₙ |
| D16S539 | 16q24-qter | anonymous | (AGAT)ₙ |
| D18S51 | 18q21.3 | anonymous | (AGAA)ₙ |
| D21S11 | 21q11.2-q21 | anonymous | (TCTA)ₙ |
| | | | (TCTG)ₙ |
| | | | [(TCTA)₃ TA |
| | | | (TCTA)₃ TCA |
| | | | (TCTA)₂ TCCA |
| | | | TA] (TCTA)ₙ |

Polymerase Chain Reaction

PCR is based on the use of two specific synthetic oligonucleotides which are used as primers in the PCR reaction to obtain one or more DNA fragments of specific lengths. The test can detect the presence of as little as one DNA molecule per sample, giving the characteristic DNA fragment. Polymerase chain reaction (PCR): a technique in which cycles of denaturation, annealing with primer, and extension with DNA polymerase are used to amplify the number of copies of a target DNA sequence by >10⁶ times.

In general, PCR can be performed according to the following protocol (adapted from U.S. Pat. No. 4,683,195). The specific nucleic acid sequence is produced by using the nucleic acid containing that sequence as a template. If the nucleic acid contains two strands, it is necessary to separate the strands of the nucleic acid before it can be used as the template, either as a separate step or simultaneously with the synthesis of the primer extension products. This strand separation can be accomplished by any suitable denaturing method including physical, chemical or enzymatic means. One physical method of separating the strands of the nucleic acid involves heating the nucleic acid until it is completely (>99%) denatured. Typical heat denaturation can involve temperature ranging from about 80 degrees to 105 degrees Celcius for times ranging from about 1 to 10 minutes. Strand separation can also be induced by an enzyme from the class of enzymes known as helicases or the enzyme RecA, which has helicase activity and in the presence of riboATP is known to denature DNA. The reaction conditions suitable for separating the strands of nucleic acids with helicases are described by Cold Spring Harbor Symposia on Quantitative Biology, Vol. XLIII “DNA: Replication and Recombination” (New York: Cold Spring Harbor Laboratory, 1978), B. Kuhn et al., “DNA Helicases”, pp. 63-67, and techniques for using RecA are reviewed in C. Radding, Ann. Rev. Genetics, 16:405-37 (1982). If the original nucleic acid constitutes the sequence to be amplified, the primer extension product(s) produced will be completely complementary to the strands of the original nucleic acid and will hybridize therewith to form a duplex of equal length strands to be separated into single-stranded molecules.

When the complementary strands of the nucleic acid or acids are separated, whether the nucleic acid was originally double or single stranded, the strands are ready to be used as a template for the synthesis of additional nucleic acid strands. This synthesis can be performed using any suitable method. Generally it occurs in a buffered aqueous solution, preferably at a pH of 7-9, most preferably about 8. Preferably, a molar excess (for cloned nucleic acid, usually about 1000:1 primer: template, and for genomic nucleic acid, usually about 10^{6:}1 primer:template) of the two oligonucleotide primers is added to the buffer containing the separated template strands. It is understood, however, that the amount of complementary strand can not be known if the process herein is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP and TTP are also added to the synthesis mixture in adequate amounts and the resulting solution is heated to about 90 degrees-100 degrees Celsius for from about 1 to 10 minutes, preferably from 1 to 4 minutes. After this heating period the solution is allowed to cool to from 20 degrees-40 degrees Celsius, which is preferable for the primer hybridization. To the cooled mixture is added an agent for polymerization, and the reaction is allowed to occur under conditions known in the art. This synthesis reaction can occur at from room temperature up to a temperature above which the agent for polymerization no longer functions efficiently. Thus, for example, if DNA polymerase is used as the agent for polymerization, the temperature is generally no greater than about 45 degrees. C. An amount of dimethylsulfoxide (DMSO) can be present which is effective in detection of the signal or the temperature is 35 degrees-40 degrees Celsius. In one aspect of the invention, 5-10% by volume DMSO is present and the temperature is 35 degrees-40 degrees Celsius. For certain applications, where the sequences to be amplified are over 110 base pair fragments, an effective amount (e.g., 10% by volume) of DMSO is added to the amplification mixture, and the reaction is carried out at 35 degrees-40 degrees Celsius, to obtain detectable results or to enable cloning.

The agent for polymerization can be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, reverse transcriptase, and other enzymes, including heat stable enzymes, which will facilitate combination of the nucleotides in the proper manner to form the primer extension products which are complementary to each nucleic acid strand. Generally, the synthesis will be initiated at the 3′ end of each primer and proceed in the 5′ direction along the template strand, until synthesis terminates, producing molecules of different lengths. There can be agents, however, which initiate synthesis at the 5′ end and proceed in the other direction, using the same process as described above.

The newly synthesized strand and its complementary nucleic acid strand form a double-stranded molecule which is used in the succeeding steps of the process. In the next step, the strands of the double-stranded molecule are separated using any of the procedures described above to provide single-stranded molecules.

New nucleic acid is synthesized on the single-stranded molecules. Additional inducing agent, nucleotides and primers can be added if necessary for the reaction to proceed under the conditions prescribed above. Again, the synthesis will be initiated at one end of the oligonucleotide primers and will proceed along the single strands of the template to produce additional nucleic acid. After this step, half of the extension product will consist of the specific nucleic acid sequence bounded by the two primers.

The steps of strand separation and extension product synthesis can be repeated as often as needed to produce the desired quantity of the specific nucleic acid sequence. As will be described in further detail below, the amount of the specific nucleic acid sequence produced will accumulate in an exponential fashion.

When it is desired to produce more than one specific nucleic acid sequence from the first nucleic acid or mixture of nucleic acids, the appropriate number of different oligonucleotide primers are utilized. For example, if two different specific nucleic acid sequences are to be produced, four primers are utilized. Two of the primers are specific for one of the specific nucleic acid sequences and the other two primers are specific for the second specific nucleic acid sequence. In this manner, each of the two different specific sequences can be produced exponentially by the present process. The polymerase chain reaction process for amplifying nucleic acid is covered by U.S. Pat. Nos. 4,683,195, 4,965,188 and 4,683,202 and European patent Nos. EP 201184 EP 200362.

DNA samples are subjected to PCR amplification using primers and thermocycling conditions specific for each locus that contains the STR of interest. In one example, the primers are selected from the group shown in Table 2. The specific amplification procedures and primer sequences relating to each locus and allelic ladder, as well as a description of locus-specific primers are described in U.S. Pat. Nos. 6,156,512 and 5,192,659.

TABLE 2

REPRESENTATIVE PRIMERS FOR SIX OF THE THIRTEEN CODIS STR LOCI

| | | | | |
|---|---|---|---|---|
| -D16S539 | primer 1: | GGG GGT CTA AGA GCT TGT AAA AAG | | 1 |
| | primer 2: | TGT GCA TCT GTA AGC ATG TAT CTA TC | 2 | |
| -D7S820 | primer 1: | GAA CAC TTG TCA TAG TTT AGA ACG | | 3 |
| | primer 2: | GCC CAA AAA GAC AGA CAG AA | 4 | |
| -D13S317 | primer 1: | ACA GAA GTC TGG GAT GTG GA | | 5 |
| | primer 2: | GCC CAA AAA GAC AGA CAG AA | 6 | |
| -D5S818 | primer 1: | GGG TGA TTT TCC TCT TTG GT | | 7 |
| | primer 2: | TGA TTC CAA TCA TAG CCA CA | 8 | |
| -D7S820 | primer 1: | ATG TTG GTC AGG CTG ACT ATG | | 9 |
| | primer 2: | CCA CAT TTA TCC TCA TTG ACA G | 10 | |
| -D7S820 | primer 1: | ATG TTG GTC AGG CTG ACT ATG | | 11 |
| | primer 2: | TCC ACA TTT ATC CTC ATT GAC AG | 12 | |
| -D5S818 | primer 1: | GGG TGA TTT TCC TCT TTG GTA TCC | | 13 |
| | primer 2: | AGT GAT TCC AAT CAT AGC CAC AG | 14 | |

In one embodiment, the DNA samples can be amplified simultaneously at the loci CSF1PO, TPOX, TH01, vWA, D5S818, D7S820, D13S317, and D16S539 using the GenePrint™ PowerPlex™ 1.1 System (Promega, Madison, Wis., USA) (i.e., PowerPlex kit) and a GeneAmp® PCR System 9600 DNA Thermal Cycler (PE Biosystems, Foster City, Calif., USA). The GenePrint PowerPlex 1.1 System contains all reagents for the PCR except the Taq DNA polymerase. Taq or AmpliTaq Gold™ (PE Biosystems) can be used in the PCR. One of the primers for each of the loci D5S818, D7S820, D13S317, and D16S539 is labeled with fluorescein, and for the loci CSF1PO, TPOX, TH01, and vWA one primer for locus is labeled with carboxy-tetramethylrhodamine. The GenePrint PowerPlex 2.1 System enables simultaneous amplification of 9 STR loci. One of the primers for each of the loci Penta E (a pentanucleotide repeat locus), D18S51, D21S10, TH01, and D3S1358, is labeled with fluorescein, and for the loci FGA, TPOX, D8S1179, and vWA the primer is labeled with carboxy-tetramethylrhodamine. Thus, the 13 core STR loci for CODIS can be amplified using the GenePrint PowerPlex 1.1 and GenePrint PowerPlex 2.1 Systems.

Polyacrylamide Gel Electrophoresis

The process for typing the amplified STRs entails separating the fragments, usually by polyacrylamide gel electrophoresis (Sambrook et al. (1989)), and detecting the products after separation has been completed. The electrophoretic gel can contain a denaturant so that the amplified products are separated as single-stranded molecules. Better separation of the STR alleles can be achieved using denaturing gel electrophoresis.

The use of polyacrylamide gel in electrophoresis (PAGE) allows for a separation or fractionation of samples on the basis of molecular size in addition to the charge differences. The separation by size is the result of the sieving effect imparted by control of the gel pore size in a “separating gel” layer.

The gels can consist of two separately polymerized layers of polyacrylamide, the separating and the stacking gel. The polymer is the result of reaction between monomer and co-monomer or cross-linking agent (percent C). The sum of the concentrations of acrylamide monomer and cross-linking agent is expressed as percent T. The separating gel has a higher concentration of monomers and consequently a smaller pore size. The actual separation of the samples takes place in this gel. The restriction created by the small pores of this gel endows PAGE with high resolution power. There can be a second gel layer with larger pore size or stacking gel to help the sample concentrate itself into tightly-packed starting zones.

The gels are placed in an electrophoretic chamber containing electrolyte buffer. The sample, generally combined with a high-density solution and a tracking dye, is placed between the gel and the buffer. The high-density solution helps the sample diffuse less. The tracking dye helps to visually follow the progress of the electrophoresis and also functions as a reference point for the measurement of the relative mobility of the bands (R_{f}). Upon application of an electrical potential, the leading ion of the separating compartment, which is chosen to have a higher effective mobility than the sample species, migrates out in front of all others, while the trailing ion of the electrolyte buffer replaces it, both moving in the same direction. Behind the leading zone other zones form, depending on the specific mobilities of the sample species, and produce discrete bands. The buffer ions and pH are very critical to the good resolution of the macromolecular mixture to be separated and to the enzymatic activity remaining after the electrophoretic separation has occurred.

Discontinuous (disc) electrophoresis utilizing polyacrylamide as the supporting medium has been claimed as one of the most effective methods for the separation of ionic components. As the name indicates, it employs discontinuous (multiphasic) buffers varying in chemical composition and properties on electrode wells and gels. The theory of discontinuous buffers was introduced by Omstein and Davis [Ann. N.Y. Acad. Sci., 121:320 and 404 (1964)].

Following electrophoretic separation and visualization of amplified alleles, individual DNA samples containing potential ladder alleles can be identified to analyze STR fragments. Samples are selected based upon the expected band separation for molecular weight differences corresponding to integral numbers of repeat units. Following the construction of allelic ladders for individual loci, they can be mixed and loaded for gel electrophoresis at the same time as the loading of amplified samples occurs. Each allelic ladder co-migrates with alleles in the sample from the corresponding locus. Such techniques are described in U.S. Pat. No. 6,221,598 to Schumm and U.S. Pat. No. 6,156,512 to Schumm.

Detection of Polymorphic STRs

After electrophoresis, the separated amplified products can be stained using a general stain, such as silver or by labeling the primers with a fluorescent tag (so that the tag will be incorporated into the amplified products during the PCR). After electrophoresis, the gel is removed from the electrophoresis apparatus and subsequently scanned using a fluorescent scanner. This detection platform is equipped with a laser, filters, and an emission detection device. Silver staining is also generally well-known to the art. Somerville and Wang (1981) and Boulikas and Hancock (1981) first described the detection of nucleic acids using a silver staining process. Bassam et al. (1991) describe a silver staining protocol for polymerase chain reaction (PCR) amplified DNA fragments.

Individual DNA samples containing amplified alleles can be compared with a size standard such as a DNA marker or locus-specific allelic ladder to determine the alleles present at each locus within the sample. Allelic ladders are constructed for STR loci with the goal of including several or all known alleles with lengths corresponding to amplified fragments containing an integral number of copies of polymorphic sequences. The DNA is then visualized by any number of techniques, including silver staining, radioactive labeling, or fluorescent labeling (Bassam et al. (1991)), various dyes or stains with denaturing or native gel electrophoresis using any available gel matrix or size separation method.

In another embodiment of the present invention the differential label for each specific sequence is selected from the group consisting of fluorescers, radioisotopes, chemiluminescers, enzymes, stains and antibodies. One specific embodiment uses the fluorescent compounds Texas Red, tetramethylrhodamine-5-(and-6) isothiocyanate, NBD aminoheanoic acid and fluorescein-5-isothiocyanate.

Multicolor detection enables an increase in the number of loci that can be analyzed simultaneously. Loci of similar size (that superimpose each other) can be resolved if labeled with different colored fluors, if the scanning/detector device is capable of separating the fluors, if the scanning/detector device is capable of separating the fluor emissions. These fluors are compatible with the FMBIO II fluorescent scanner (Hitachi Genetic Systems/MiraiBio, Alameda, Calif., USA), which is used to detect the separated amplified products.

In many cases, the selected amplified alleles are subjected to sequence analysis to confirm the sequence heterogeneity among various alleles. The DNA sequencing technique of Sanger et al. (1977), an enzymatic dideoxy chain termination method can be employed. Traditional methods of DNA sequencing utilize a radiolabeled oligonucleotide primer or the direct incorporation of a radiolabeled nucleotide. Fluorescent labeled oligonucleotide primers can be used in place of radiolabeled primers for sensitive detection of DNA fragments (U.S. Pat. No. 4,855,225 to Smith et al.). Chapter 13 of Sambrook, J. et al. (1989) describes DNA sequencing in general, as well as various DNA sequencing techniques.

DNA Amplification and Typing

The first post-PCR typing approach used for forensic purposes was detection of sequence polymorphisms by use of allele-specific oligonucleotide (ASO) hybridization probes in a dot blot format. Under appropriate conditions, ASO probes hybridize only to DNA sequences that contain their exact complement. Thus, a different ASO probe is required for each allele to be detected at a locus. A battery of ASO probes is bound to a nylon membrane strip. The configuration where ASO probes are immobilized on a support, instead of amplified DNA, is known as a reverse dot blot format. The strip can accommodate probes for multiple alleles at several loci. The corresponding regions of DNA are amplified by the PCR, and the amplified alleles are hybridized to the immobilized probes to which they are complementary. Because an identifier molecule (or tag) is attached to the 5′ end of one of the primers, a detectable label is incorporated into the amplified alleles. When compelled with probes at fixed locations on the nylon test strip, the amplified alleles can thus be detected and typed.

The general protocol for typing these PCR-based loci entails: extraction of DNA, amplification of specific loci with biotin-labeled primers, denaturation of the amplified products, hybridization of the denatured DNA to probes immobilized on a nylon strip, binding of streptavidin-horseradish peroxidase substrate to the biotin molecules, and detection of allelic products using a calorimetric substrate.

Typing of the HLA-DQA1 locus is a well characterized PCR-based system using the reverse dot blot format for the analysis of forensic specimens. The HLA-DQA1 protein is a heterodimer composed of one alpha chain (encoded by the HLA-DQ alpha locus) and one beta chain. It is expressed in B-lymphocytes, macrophages, thymic epithelium, an activated T-cells. The HLA-DQ protein serves as an integral membrane protein for binding, as well as for presenting, antigen peptide fragments to the T-cell receptor of CD4+T hymphocytes. The polymorphism, which determines the HLA DQA1 alleles, is detected by amplification and hybridization to the test strip of a 242-bp fragment (or 239-bp length for alleles 2 and 4) from the second exon of the HLA-DQ alpha gene. Eight common alleles have been identified; they are designated 1.1, 1.2, 1.3, 2, 3, 4.1, 4.2, and 4.3. A kit is commercially available (AmpliType® PM+DQA1 PCR Amplification and Typing Kit; PE Biosystems) for typing the HLA-DQA1 locus.

Four probes are designed to detect alleles 1, 2, 3, and 4; the 1 allele can be subtyped further as a 1.1, 1.2, or a 1.3 allele, and the 4 allele can be subtyped as a 4.1 or a 4.2/4.3 (the 4.2 and 4.3 alleles cannot be distinguished with the kit). All of the probes for detecting these alleles are contained on a single strip.

The molecular tag attached to one of the HLA-DQA1 primers to detect the amplified allele-probe hybrid complex can be biotin. Following hybridization, a streptavidin-horseradish-peroxidase complex is allowed to bind with biotin. The horseradish peroxidase then oxidizes a substrate, such as tetramethyl-benzidine (TMB), which results in a blue precipitate at the hybridization site that indicates the presence of specific alleles.

While the ability to type very small quantities of DNA is possible at the HLA-DQA1 locus, polymorphic data from a single locus does not achieve the power of discrimination provided by RFLP typing of VNTR loci. To increase the discrimination power of PCR-based DNA analyses, the Ampli-Type PM+DQA1 PCR Amplification and Typing Kit also allows for the simultaneous amplification (i.e., multiplex) of the HLA-DQA1 locus and that of five other genetic markers—LDLR, GYPA, HBGG, D7S8, and Gc.

The LDLR, GYPA, HBGG, D7S8, and Gc loci [PolyMarker (PE Biosystems) or PM loci] are typed simultaneously, also using ASO probes by reverse dot blot analysis, in a manner similar to that of HLA-DQA1. LDLR, GYPA, and D7S8 each have two detectable alleles (designated A and B), while HBGG and Gc each have three alleles that can be typed (designated A, B, and C). This can be achieved via a multiplex system, such as the DQA1+PM system.

Further detailed description and examples of such methods are disclosed in Budowle et al. (DNA Typing Protocols: Molecular Biology and Forensic Analysis, Eaton Publishing: MA, USA (2000)).

Other methods to carry out DNA typing will be readily apparent to one skilled in the art, including, but not limited to:

(1) Hybridization-based techniques, selected from the group, including but not limited to: Multi-locus minisatellite fingerprinting (Jeffreys et al., 1985), Oligonucleotide fingerprinting (Ali et al. 1986; Weising et al. 1991), Restriction fragment length polymorphism (RFLP) (Wyman and White 1980, Botstein et al. 1980)

(2) Amplification-based nucleic acid scanning techniques, selected from the group including, but not limited to: Random amplified polymorphic DNA (RAPD) (Williams et al. 1990), Arbitrarily primed PCR (AP-PCR) (Welsh and McClelland 1990), DNA amplification fingerprinting (DAF) (Caetano-Anollés et al. 1991), Minihairpin primer-driven DAF (mhpDAF) Caetano-Anollés and Gresshoff 1994), Arbitrary signatures from amplification profiles (ASAP) (Caetano-Anollés and Gresshoff 1996), AFLP (Vos et al. 1995), Alu-PCR (Nelson et al. 1989) rep-PCR (Versalovic et al. 1994), Microsatellite-primed PCR (MP-PCR) (Meyer et al. 1993; Perring et al. 1993), Anchored MP-PCR (AMP-PCR) (Zietkiewicz et al. 1994), Random amplified microsatellite polymorphism (RAMP) (Wu et al. 1994), Random amplified hybridization microsatellites (RAHM), (Cifarelli et al. 1995, Richardson et al. 1995; Ender et al. 1996), Nucleic acid scanning-by-hybridization (NASBH) (Salazar and Caetano-Anollés 1996), RAPD dot-blot hybridization (Penner et al. 1996), Differential display reverse transcription (DDRT) PCR (Liang and Pardee 1992), RNA arbitrarily primed PCR (RAP-PCR) (Welsh et al. 1992), cDNA-AFLP (Bachem et al. 1996).

(3) Amplification-based nucleic acid profiling techniques selected from the group consisting of, but not limited to: Amplified fragment length polymorphism (AmpFLP) (Jeffreys et al. 1988, Horn et al. 1989; Boerwinkle et al. 1989), Minisatellite variant repeat PCR (MVR-PCR) (Jeffreys et al. 1991), Simple sequence repeat PCR(SSR-PCR) (Litt and Luty 1989, Weber and Can 1989, Tautz 1989).

(4) Sequence-targeted techniques selected from the group including, but not limited to Allele specific oligonucleotide (ASO) hybridization (Saiki et al. 1986), TaqMan ASO (Livak et al. 1995), Allele specific reverse dot blot hybridization (Keller et al. 1991), Single strand conformation polymorphism (SSCP) (Orita et al. 1989), Cleaved amplified polymorphic sequence (CAPS) analysis (Konieczny and Ausubel 1993), Coupled amplification and sequencing (CAS) (Ruano and Kidd 1991), Amplification refractory mutation system (ARMS) (Newton et al. 1989), Oligonucleotide ligation assay (OLA) (Landegren et al. 1988, Nickerson et al. 1990), Coupled amplification and oligonucleotide ligation (CAL) (Eggerding 1995), Genetic bit analysis (GBA) (Nikiforov et al. 1994), Oligonucleotide arrays (reviewed in Southern 1996)

V. Kits for the Extraction of DNA

The invention includes a kit for the separation of male and female DNA that can include (i) wells with filters that are larger than DNA and smaller than unlysed cells, and (ii) reagents for the selective lysis of female cells followed by the lysis of male sperm cells. Alternately, the kit can include (i) wells with filters that are larger than DNA and smaller than unlysed cells, and (iii) an instruction manual to teach the user how to use the kit for the separation of male and female DNA. The kit may also include (i) wells with filters that are larger than DNA and smaller than unlysed cells, (ii) reagents for the selective lysis of female cells followed by the lysis of male sperm cells, and, optionally, (iii) an instruction manual to teach the user how to use the kit for the separation of male and female DNA.

In one embodiment, the kit can include containers which contain the reagents for DNA extraction. The reagents can be selected from the group, including, but not limited to sodium dodecyl sulfate (SDS), Proteinase K, and dithiothreitol (DTT) or any other agent that cleaves disulfide bonds and Proteinase K. In a specific embodiment, the filters within the kit contain pores that are larger than cell lysate, including DNA and smaller than spermatozoa. In a particular embodiment, since sperm cell heads are typically about 25 microns, the pore size of the filter is less than 5-10 microns.

The present invention is described in further detail in the following examples. These examples are intended to be illustrative only, and are not intended to limit the scope of the invention.

### EXAMPLES

### Example 1

### Extraction of Spermatozoa DNA from a Cellular Mixture, Comprising Epithelial and Sperm Cells Deposited on a Substrate

A biological specimen including an epithelial cell and sperm cell mixture deposited on a substrate is obtained from a crime scene. The specimen, typically a vaginal/cervical swab, is placed in one of the 96 wells of a plate, for example the Qiafilter™ 96 well plate. The plate is then placed on a 96 well collection block. To the well containing the substrate, 500 μl of Differential Extraction Buffer I (80% TNE, 1% Sarkosyl) and 5 μl of Proteinase K (20 mg/ml) is added. The plate is then covered by a tape sheet and incubated at 37° C. for 2 hours. After incubation, the plate is centrifuged for 3 minutes at 5,600×g. The 96 well collection block is then removed and labeled as the non-sperm fraction. This can be placed in the refrigerator until ready for DNA purification. The plate is placed on a new 96 well collection block (2 ml well volume capacity). The tape sheet is removed and 500 μl of Differential Extraction Buffer I and 5 μl of Proteinase K (20 mg/ml) is added. The plate is covered by a tape sheet and incubated at 37° C. for 1 hour. After incubation, the plate is centrifuged for 3 minutes at 5,600×g. The tape sheet is removed and 500 μl of Differential Extraction Buffer I is added. The plate is covered by a tape sheet and centrifuged for 3 minutes at 5,600×g. This step is repeated once for a final wash. To the well is then added 350 μl of Differential Extraction Buffer II (42.86% TNE, 2.86% Sarkosyl), 40 μl 0.39 M DTT, and 10 μl of Proteinase K (20 mg/ml). The plate is placed on a new 96 well collection block, covered with a tape sheet and incubated at 37° C. for 2 hours. After incubation, the plate is centrifuged for 3 minutes at 5,600×g. The plate can then be discarded and the collection block is labeled as the sperm cell fraction. The non-sperm and sperm cell fractions can then be purified using the Qiagen™ blood kit or other currently available methods.

### Example 2

### Validation of the New Technique to Sequentially Extract DNA from Cell Mixtures

In the initial experiment to determine if the method was effective, three swabs were prepared as described below:

| | | Volume of diluted semen |
|---|---|---|
| | | placed on oral swab from |
| Swab | Semen Dilution | female individual (μl) |
| 1 | 1:10 | 50 |
| 2 | 1:10 | 100 |
| 3 | 1:10 | 100 |

The diluted semen was placed on the tip of each of the swabs for consistent sampling later. The swabs were allowed to dry overnight. The tip of each swab was cut off and placed in a well of a Qiafilter™ 96 Plate. The epithelial cells and sperm cells were then separated as described above. The DNA from the non-sperm cell fraction and sperm cell fractions was then purified as described below:

Add 500 μl of an appropriate buffer to each well containing lysate. Mix with pipettor.

Cover with AirPore tape sheet and incubate at 70° C. for 10 minutes.

Add 500 μl of 100% ethanol to each well containing lysate. Mix with pipettor.

Add 750 μl of lysate mixture to appropriate well of a QIAamp 96-well plate on an S block.

After all samples have been added, cover plate with AirPore tape sheet.

Centrifuge plate at 6,000 rpm's (5,600×g) for 10 minutes.

Remove tape sheet and add remaining lysate mixture to the appropriate wells. Cover plate with AirPore tape sheet and centrifuge at 6,000 rpm's (5,600×g) for 10 minutes.

Empty S block and rinse. Add 500 μl of Buffer AW1 to each well, cover with AirPore tape sheet, and centrifuge at 6,000 rpm's (5,600×g) for 5 minutes.

Add 500 μl of an appropriate buffer to each well, cover with AirPore tape sheet, and centrifuge at 6,000 rpm's (5,600×g) for 5 minutes.

Place plate on a rack of 96 microtubes, and incubate at 70° C. for 10 minutes uncovered.

Remove the plate and rack of microtubes from the incubator and add 60 μl of Buffer AE preheated to 70° C. to each well. Cover plate with an AirPore tape sheet and incubate at 70° C. for one minute.

Remove from incubator and centrifuge at 6,000 rpm's (5,600×g) for 2 minutes.

Place strip caps on microtubes.

The DNA obtained from both fractions was then quantitated, PCR amplified at 13 STR loci using Cofiler and Profiler Plus (Applied Biosystems), and analyzed on an AB377. The resulting profiles demonstrated that the method was able to successfully separate the sperm cells from the epithelial cells. The sperm cell fraction profile did match the known profile of the semen donator.

### Example 3

### Evaluation of the New Technique for the Sequential Extraction of DNA Versus the Standard Protocol

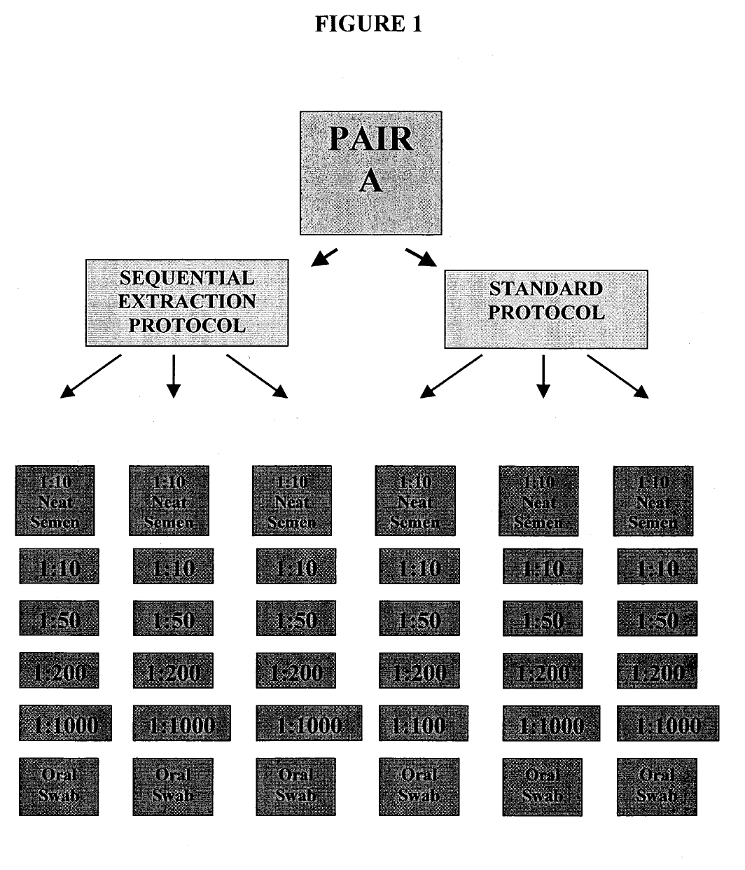

Swabs were prepared as described in FIG. 1. “Pair A” refers to a known male semen donor and oral swabs from a known female. A second set of swabs was similarly prepared for another known pair, B, for a total of 72 swabs. Thirty-swabs were then analyzed following both the current protocol and the new protocol. The new protocol was performed following the steps outlined above in Examples 1 and 2.

The standard protocol involves a single wash during the separation process and an organic extraction followed by ethanol precipitation for DNA purification. The DNA for all samples was then quantitated, PCR amplified at 13 STR loci using Cofiler and Profiler Plus (Applied Biosystems), and analyzed on an AB3100.

TABLE 1
Summary of Results from Example 3 (sperm cell fractions)

| | Average Results for Sperm Cell Fraction | |
|---|---|---|
| Sample | Current Protocol | New Protocol |
| 1:10 Neat Semen | Weak male profile | Strong male profile |
| 1:10 on oral swab | Weak male profile to | Clean male profile to |
| | strong mix with female | strong male profile with |
| | profile | occasional weak visible |
| | | female alleles |
| 1:50 on oral swab | Weak mixed results to no | Equal male/female mixed |
| | interpretable results | profiles to major male |
| | | component with minor |
| | | female component. |
| 1:200 on oral swab | Female profile, hint of | Female profile, hint of |
| | male | male |
| 1:1000 on oral swab | Female profile | Female profile |
| Oral swab | Female profile | Female profile |

The results for this experiment demonstrated a greatly increased recovery of sperm cell DNA using the new protocol compared to that of the current protocol. Also, the sperm cell fractions of the new protocol appeared to be as “clean” as or “cleaner” than similar samples processed using the current protocol.

This invention has been described with reference to illustrative embodiments. Other embodiments of the general invention described herein and modifications there of will be apparent to those of skill in the art and are all considered within the scope of the invention.

We claim:

1. A method of isolating DNA from a heterogenous mixture of cells comprising:
(a) providing a sample containing a heterogeneous mixture of cells that includes a first cell type;
(b) selectively lysing the first cell type within the mixture of cells;
(c) allowing the lysed mixture that includes the DNA from the first cell type to flow through a size exclusion filter;
(d) collecting the filtrate that contains the DNA from the first cell type.

2. A method of isolating DNA from a heterogenous mixture of cells comprising:
(a) providing a sample containing a heterogeneous mixture of cells that includes at least a first and second cell type;
(b) selectively lysing the first cell type within the mixture of cells;
(c) allowing the lysed mixture that includes DNA from the first cell type to flow through a size exclusion filter;
(d) collecting the filtrate that contains the DNA from the first cell type;
(e) separately collecting the intact heterogeneous mixture of cells that includes at least the second cell type;
(f) selectively lysing the second cell type within the mixture;
(g) allowing the lysed mixture that includes DNA from the second cell type to flow through a size exclusion filter; and
(h) collecting the filtrate that contains the DNA from the second cell type.

3. The method of claim 1 or 2 wherein the sample is selected from the group consisting of a biological, medical or forensic sample.

4. The method of claim 1 or 2 wherein the sample is a forensic sample.

5. The method of claim 4 wherein the forensic sample is obtained from a rape victim.

6. The method of claim 1 or 2 wherein the sample is deposited on a substrate.

7. The method of claim 6 wherein the substrate is a swab obtained from a rape victim.

8. The method of claim 7 wherein the swab is a vaginal swab.

9. The method of claim 1 or 2 wherein the cells include human cells.

10. The method of claim 1 or 2 wherein the cells include animal cells.

11. The method of claim 1 or 2 wherein the cells include vegetal cells.

12. The method of claim 1 or 2 wherein the first cell type is selected from the group consisting of erythrocytes, platelets, neutrophils, lymphocytes, monocytes, eosinophils, basophils, adipocytes, chondrocytes, pancreatic islet cells, thyroid cells, parathyroid cells, parotid cells, tumor cells, neuronal cells, glial cells, astrocytes, and red blood cells.

13. The method of claim 1 or 2 wherein the first cell type is selected from the group consisting of white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells and memory cells.

14. The method of claim 1 or 2 wherein the first cell type is selected from the group consisting of T cells, B cells, plasma cells, muscle cells, ovarian cells, uterine cells, prostate cells, vaginal epithelial cells, sperm cells, testicular cells, germ cells, egg cells, leydig cells, peritubular cells, sertoli cells, lutein cells, cervical cells and endometrial cells.

15. The method of claim 1 or 2 wherein the first cell type is selected from the group consisting of cells, mammary cells, follicle cells, mucous cells, ciliated cells, nonkeratinized epithelial cells, keratinized epithelial cells, lung cells, goblet cells, columnar epithelial cells, squamous epithelial cells, osteocytes, osteoblasts, osteoclasts and epithelial cells.

16. The method of claim 1 or 2 wherein the first cell type is an epithelial cell.

17. The method of claim 1 or 2 wherein the heterogeneous mixture of cells comprises at least epithelial cells and sperm cells.

18. The method of claim 1 or 2 wherein the cell lysis is achieved through mechanical disruption.

19. The method of claim 1 or 2 wherein the cell lysis is achieved through chemical treatment.

20. The method of claim 1 or 2 wherein the cell lysis is achieved through enzymatic digestion.

21. The method of claim 1 or 2 wherein the cells are lysed with a detergent.

22. The method of claim 16 wherein the detergent is selected from the group consisting of SDS, sarkosyl, Triton and TWEEN.

23. The method of claim 16 wherein the detergent is sarkosyl.

24. The method of claim 1 or 2 wherein the cells are lysed with a proteinase.

25. The method of claim 19 wherein the cells are lysed with Proteinase K.

26. The method of claim 1 or 2 wherein the cells are lysed with a detergent and a proteinase.

27. The method of claim 21 wherein the detergent is sarkosyl and the proteinase is Proteinase K.

28. The method of claim 1 or 2 wherein the filter has pores that are smaller than intact cells and larger than DNA.

29. The method of claim 1 or 2 wherein the filter has a pore size of 5 microns or less.

30. The method of claim 1 or 2 wherein the filter has a pore size of 10 microns or less.

31. The method of claim 12 wherein the filter has pores that are smaller than sperm cells and larger than DNA.

32. The method of claim 1 or 2 wherein the filter is comprised of a material that is not degraded by buffers or reagents used to lyse the cells.

33. The method of claim 1 or 2 wherein the DNA flows through the filter by gravity, centrifugation or vacuum.

34. The method of claim 2 wherein the second cell type is selected from the group consisting of erythrocytes, platelets, neutrophils, lymphocytes, monocytes, eosinophils, basophils, adipocytes, chondrocytes, pancreatic islet cells, thyroid cells, parathyroid cells, parotid cells, tumor cells, neuronal cells, glial cells, astrocytes, and red blood cells.

35. The method of claim 1 or 2 wherein the second cell type is selected from the group consisting of white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells and memory cells.

36. The method of claim 1 or 2 wherein the second cell type is selected from the group consisting of T cells, B cells, plasma cells, muscle cells, ovarian cells, uterine cells, prostate cells, vaginal epithelial cells, sperm cells, testicular cells, germ cells, egg cells, leydig cells, peritubular cells, sertoli cells, lutein cells, cervical cells and endometrial cells.

37. The method of claim 1 or 2 wherein the second cell type is selected from the group consisting of cells, mammary cells, follicle cells, mucous cells, ciliated cells, nonkeratinized epithelial cells, keratinized epithelial cells, lung cells, goblet cells, columnar epithelial cells, squamous epithelial cells, osteocytes, osteoblasts, osteoclasts and epithelial cells.

38. The method of claim 2 wherein the second cell type is sperm cells.

39. The method of claim 1 or 2 wherein cell lysis is achieved with at least dithiothreitol (DTT).

40. The method of claim 30 wherein cell lysis is achieved with at least dithiothreitol (DTT).

41. The method of claims 1, 2 or 30 wherein cell lysis is achieved with sarkosyl and DTT.

42. The method of claims 1, 2 or 30 wherein cell lysis is achieved with Proteinase K and DTT.

43. The method of claims 1, 2, or 30 wherein cell lysis is achieved with Proteinase K, sarkosyl and DTT.

44. A kit comprising (i) wells with filters that are larger than DNA and smaller than intact cells; and (ii) reagents for the selective lysis of female cells followed by the lysis of sperm cells.

45. The kit of claim 36 wherein the female cells include epithelial cells.

46. The kit of claim 36 wherein the reagents include detergents.

47. The kit of claim 36 wherein the reagents include proteinases.

48. The kit of claim 36 wherein the reagents are selected from the group consisting of sarkosyl, Proteinase K and dithiothretol (DTT).

49. The kit if claim 36 wherein the filter is removable.

50. The kit of claim 36 wherein multiple wells are attached to each other and comprise a plate.
